(19) United States
(12) Patent Application Publication
(10) Pub. No.: US 2025/0090571 A1
(43) Pub. Date: Mar. 20, 2025

(54) CARD9 ATTENUATES AB PATHOLOGY AND MODIFIES MICROGLIAL RESPONSES IN AN ALZHEIMER’S DISEASE

(71) Applicant: University of Virginia Patent Foundation, Charlottesville, VA (US)

(72) Inventors: John R. Lukens, Charlottesville, VA (US); Hannah Elizabeth Ennerfelt, Palo Alto, CA (US); Alexis M. Johnson, Cherlottesville, VA (US)

(21) Appl. No.: 18/773,077

(22) Filed: Jul. 15, 2024

Related U.S. Application Data
(60) Provisional application No. 63/583,042, filed on Sep. 15, 2023.

(51) Int. Cl. A61K 31/716; A61K 38/17; A61K 48/00; A61P 25/28; C12N 15/85

(52) U.S. Cl. CPC A61K 31/716; A61K 38/1709; A61K 48/005; A61P 25/28; C12N 15/85

(57) ABSTRACT

Provided herein are methods and compositions to enhance or activate protective microglial activities (such as phagocytosis of neurotoxic material) by activating CARD9.

### PRIORITY

This patent application claims the benefit of priority to U.S. Provisional Application Ser. No. 63/583,042, filed Sep. 15, 2023, which is incorporated by reference herein in its entirety.

### GOVERNMENT SUPPORT

This invention was made with Government support under Grant No. RFIAG071996 awarded by the National Institute of Health (NIH/NIA). The Government has certain rights in this invention.

### INCORPORATION BY REFERENCE OF SEQUENCE LISTING

This application contains a Sequence Listing which has been submitted electronically in ST26 format and hereby incorporated by reference in its entirety. Said ST26 file, created on Aug. 22, 2024, is named 1036369US1.xml and is 5,166 bytes in size.

### BACKGROUND

Neurodegenerative disorders, such as Alzheimer's disease (AD), are major public health issues that are likely to increase in prevalence with the aging population. In general terms, neurodegenerative diseases are thought to be driven by the accumulation of neurotoxic material such as amyloid beta (Aβ) or myelin debris in the central nervous system (CNS) (Nussbaum and Ellis, 2003; Trapp and Nave, 2008). The buildup of neurotoxic agents is believed to cause neuronal damage and death, which can ultimately lead to various forms of neurological dysfunction that include cognitive decline, motor abnormalities, mental disorders, and loss of inhibition (Chung et al., 2018; Taylor et al., 2002; Vickers et al., 2009).

### SUMMARY OF THE INVENTION

Although significant progress in recent years has been made in defining key innate immune receptors involved in Alzheimer's disease (AD), our knowledge of the specific intracellular signaling molecules that coordinate immune responses in AD remains poorly defined. Provided herein, a previously undescribed role for the innate immune signaling molecule CARD9 in an amyloid beta (Aβ)-mediated model of AD identified. It is demonstrated that CARD9 deletion in the 5×FAD mouse model of AD leads to impaired control of Aβ, worsened cognitive decline, and aberrant microglial activation. It is further shown that pharmacological activation of CARD9 provides a strategy to boost Aβ clearance from the hippocampus. Collectively, these findings uncover a previously uncharacterized molecular signaling molecule used by the innate immune system in Aβ-mediated neurological disease, and establish CARD9 as a novel molecular player that can be targeted in AD.

Provided herein is a method to enhance or activate protective microglial activities (such as phagocytosis of neurotoxic material) or reduce Aβ coverage and plaque volume compared to an untreated control comprising administering to a subject in need thereof an activator of CARD9; a method to preserve neuronal health or prevent neuronal loss comprising administering to a subject in need thereof an activator of CARD9; and a method to prevent a decrease or generate an increase in spatial learning and/or memory comprising administering to a subject in need thereof an activator of CARD9.

In some embodiments, CARD9 activator is a ligand for CLEC7A. In other embodiments, the CARD9 activator comprises a β-1,3-linked or a β-1,6-linked glucan, such as pustulan. In some embodiments, the subject has a demyelinating neuroinflammatory disease. In several embodiments, the subject has is Alzheimer's Disease (AD) or Multiple Sclerosis (MS).

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1F. CARD9 deletion leads to increased Aβ accumulation in 5×FAD mice. (A-F) Brains were harvested from 5-month-old 5×FAD, Card9^{+/-}5×FAD, and Card9^{-/-}5×FAD mice to evaluate Aβ load. (A) Representative images from sagittal sections of Aβ (D54D2, red; DAPI, blue) staining in the cortex, hippocampus, and thalamus. Original magnification: 10×; scale bar=2000 μm and 10000 μm. (B) Quantification of percent area covered by Aβ in the cortex, hippocampus, and thalamus. Combined data from three independent experiments. (C-D) Aβ levels detected by Aβ-42 ELISA. (C) Soluble (PBS buffer extraction) and (D) insoluble (guanidine extraction) fractions from 5-month-old 5×FAD, Card9+/−5×FAD, and Card9^{+/-}5×FAD half-brain hemispheres. (E-F) Representative images and quantification of Aβ plaques measuring ThioS+ (pink) plaque numbers in the cortex field of view (FOV), with combined data from a total of 50-100 plaques from 3 matching brain sections per mouse. Original magnification: 63×; scale bar=40 μm. Statistical significance between experimental groups was calculated by one-way ANOVA with Tukey's post hoc test (B-D, F). *P<0.05, **P<0.01, ***P<0.001. Error bars represent mean±S.E.M. and each data point represents an individual mouse (B-D. F).

FIGS. 2A-2H. Card9 deficiency leads to worsened neuronal health and cognitive impairment in 5×FAD mice. (A-B) Brains were harvested from 5-month-old 5×FAD. Card9+/−5×FAD, and Card9^{-/-}5×FAD mice to evaluate neuronal death. The CA1 region of the hippocampus was evaluated for neuronal cell death by the TUNEL assay (pink), NeuN staining (green) and DAPI (blue). Original magnification: 63×; scale bars=40 μm. (C—H) The Morris water maze (MWM) test was used to assess spatial learning and memory in 4-month-old 5×FAD, Card9+/−5×FAD, and Card9^{-/-}5×FAD mice. (C-G) Acquisition stage of learning in the MWM. (C) Latency to platform (acquisition). (D) Representative heatmaps of mouse trajectory on day 4 of acquisition and (E) plotted percentage of allocentric navigation strategy during MWM acquisition. (F) Distance traveled averaged from all four trials in maze (cm) and (G) average speed of travel (cm/s) on day 4 of acquisition. (H) Percentage of time spent in the target quadrant (probe). Statistical significance between experimental groups was calculated by repeated-measures two-way ANOVA with Bonferroni's post hoc test (C, E) or one-way ANOVA with Tukey's post hoc test (B, F-H) from three independent experiments. *P<0.05, **P<0.01, ***P<0.001. Error bars represent mean±S.E.M. (B. C, E-H) and each data point represents an individual mouse (B, F-H), or the average of experimental mice per group (C,E).

FIGS. 3A-31. The loss of Card9 leads to altered microgliosis in 5×FAD mice. (A-I) Brains were harvested from 5-month-old 5×FAD, Card9^{+/+}5×FAD, and Card9′5×FAD mice to assess microglial activity and Aβ plaque volume. (A-B) Representative images and quantification of microglia numbers (IBA1, cyan) surrounding ThioS⁺ plaques (pink) in the field of view (FOV) of the frontal cortex of 5×FAD, Card9^{+/-}5×FAD, and Card9′ 5×FAD mice. Original magnification: 63×; scale bar=40 μm. (C-D) Representative images of microglial proliferation measured by evaluating Ki67 (yellow) colocalization with IBA1+ (cyan) microglia in the cortex of matched sagittal sections. Original magnification: 63×; scale bar=40 μm. (E-G) Representative images and quantification of ThioS-labeled (pink) plaque sphericity and volume in the cortex. Original magnification: 63×; scale bar=20 μm. (F) Quantification of plaque sphericity with 1.00 being the most spherical. (G) Quantification of plaque volume in the cortex. (H-I) Microglial morphology calculated by Sholl analysis from a total of 12 microglia from 3 matching brain sections per mouse (n=5 mice per group). (H) Representative microglia renderings and (I) Sholl analysis. Original magnification: 63×; scale bar=10 μm. Statistical significance between experimental groups was calculated by one-way ANOVA with Tukey's post hoc test (B-D, F-G) or repeated-measures two-way ANOVA with Bonferroni's post hoc test (I). *P<0.05, **P<0.01, ***P<0.001, ****P<0.0001. Error bars represent mean±S.E.M. (B-D, F-G, I). Each data point represents an individual mouse (B-D, F-G) or the average of 5 mice (I). Data was collected from 6 fields of view (FOV) from a total of 3 matched sagittal sections (B, D) or 50-100 plaques from the cortex of each mouse (F, G).

FIGS. 4A-4K. Card9-dependent microglial regulation in 5×FAD mice. (A-C) RNA-Seq was performed on microglia from 5-month-old Card9^{+/+} (denoted as WT), Card9^{-/-}, 5×FAD, and Card9^{-/-}5×FAD mice sorted from single-cell brain suspensions using anti-CD11b⁺-coated magnetic beads and magnetic column sorting. (A) Principal component (PC) analysis of sample clustering. (B) Volcano plot comparing differentially expressed genes (FDR<0.1) between Card9^{-/-}5×FAD and 5×FAD microglia, where 4 genes are significantly downregulated, and 4 genes are upregulated. (C) Heatmap representation of the top 10 overall upregulated and downregulated genes between microglia isolated from Card9^{-/-}5×FAD and 5×FAD mice. (D) qPCR validation of Klf4 expression in microglia sorted from Card9^{+/-}5×FAD and 5×FAD mice. (E-F) Brains were harvested from 5-month-old 5×FAD, Card9^{+/-}5×FAD, and Card9^{-/-}5×FAD mice to assess microglial Ferritin Heavy Chain levels in the cortex. (E) Representative images and (F) quantification of Ferritin Heavy Chain (FHC, yellow) within microglia (IBA1, cyan) surrounding ThioS⁺ plaques (pink) in the field of view (FOV) of the frontal cortex of 5×FAD, Card9^{+/-}5×FAD, and Card9^{-/-}5×FAD mice. Original magnification: 63×; scale bar=30 ρm. (G-K) 5×FAD and Card9^{-/-}5×FAD mice fed normal or PLX5622 chow beginning at 1.5 months of age and harvested at 4 months of age. (G-H) Representative images and quantification of microglia numbers (IBA1, cyan) surrounding ThioS⁺ plaques (pink) in the FOV of the frontal cortex of normal chow- and PLX5622 chow-fed 5×FAD and Card9^{-/-}5×FAD mice. (G, I) Representative images and quantification of Aβ plaques measuring ThioS⁺ (pink) plaque numbers in the cortex FOV, with combined data from a total of 30-75 plaques from 3 matching brain sections per mouse. Original magnification: 63×; scale bar=30 μm. (J-K) Representative images and quantification of percent area covered by Aβ in the cortex from sagittal sections of normal chow-and PLX5622 chow-fed 5×FAD and Card9^{-/-}5×FAD mice (D54D2, red; DAPI, blue). Original magnification: 10×; scale bar=2000 μm. Statistical significance between experimental groups was calculated by unpaired two-tailed Student's t-test (D), one-way ANOVA with Tukey's post hoc test (F), and two-way ANOVA with Tukey's post hoc test (H-I, K). *P<0.05, **P<0.01, ***P<0.001, ****P<0.0001. Error bars represent mean±S.E.M. (D, F, H-I, K). Each data point represents an individual mouse (D, F, H-J, K).

FIGS. 5A-5H. Pustulan treatment enhances Aβ clearance in a CARD9-dependent manner in the hippocampus of 5×FAD mice. (A-H) Right and left hippocampal injection of vehicle or pustulan, respectively, into 2-month-old 5×FAD and Card9^{-/-}5×FAD mice. Brains were then harvested 7 days post injection (dpi). (A) Experimental design schematic. (B) Representative images of Aβ (pink) coverage in 2-month-old 5×FAD and Card9^{-/-}5×FAD mice 7 days following intrahippocampal injection of vehicle or 2 μg of pustulan. (C—H) Quantification of Aβ (D54D2, pink) in the hippocampus of (C-E) 5×FAD and (F-H) Card9^{-/-}5×FAD mice. Statistical significance between experimental groups was calculated by paired Student's t-test (C—H). ns=nonsignificant, *P<0.05. Representative data from 2 independent experiments (C—H). Error bars represent mean ±S.E.M. and each data point represents an individual mouse.

FIGS. SIA-SIE. Deletion of Card9 in the absence of Aβ-mediated pathology does not impact learning or memory. (A-F) The Morris water maze (MWM) test was used to assess spatial learning and memory in 4-month-old Card9^{+/+} (denoted as WT), Card9^{+/-}, and Card9^{-/}-mice. (A-E) Acquisition stage of learning in the MWM. (A) Latency to platform (acquisition). (B) Representative heatmaps of mouse trajectory on day 4 of acquisition and (C) plotted percentage of allocentric navigation strategies during MWM acquisition. (D) Distance traveled in maze (cm) and (E) average speed of travel (cm/s) on day 4 of acquisition. (F) Percentage of time spent in the target quadrant (probe). Statistical significance between experimental groups was calculated by repeated-measures two-way ANOVA with Bonferroni's post hoc test (A, C) or one-way ANOVA with Tukey's post hoc test (D-F) from three independent experiments. NS=not significant, *P<0.05. Error bars represent mean±S.E.M. (A, C-F) and each data point represents an individual mouse (D-F).

FIGS. S2A-S2F. CARD9-deficient mice do not display impaired Aβ clearance in vitro or in vivo. (A-C) Bone marrow-derived macrophages (BMDMs) from C57BL/6J or Card9-deficient mice were stimulated with 10 μM oligomeric Aβ (D54D2, red) tagged with pH sensitive CypHer5e dye (yellow) for 24 hours. (A) Representative images of stimulated BMDMs. (B) The percent area of CypHerSe dye relative to Aβ and (C) percent area of Aβ. Original magnification: 40×; scale bar =40 μm. (D-F) Brains harvested from C57BL/6J or Card9-deficient mice stereotaxically injected with Aβ (D54D2, red) tagged with pH sensitive CypHerSe dye (blue) in the cortex and harvested 48 hours post injection. (D) Representative images of the Aβ-CypHer5E injection site in the cortex of C57BL/6J and Card9-deficient mice. (E) The percentage of the ratio of CypHer5e dye and residual Aβ and (F) the percent area of IBA1+ cells at the site of injection. Consecutive sections, spaced 40 μm apart, were quantified and averaged for each mouse in a blinded fashion to assess total clearance of CypHer5E-labelled Aβ oligomers over the entire area of the injection site. Original magnification: 40×; scale bar=100 μm. Statistical significance between experimental groups was calculated by unpaired two-tailed Student's t-test from two independent experiments. Error bars represent mean±S.E.M. and each data point represents an individual mouse (B-C, E-F).

FIGS. S3A-S3E. Card9 deficiency in the absence of Aβ-mediated neuropathology does not affect microglial number or morphology in adult mice. Brains were harvested from 5-month-old Card9^{+/+} (denoted as WT), Card9^{+/+}, and Card9^{-/-} mice to assess microglial activity. (A-B) Representative images and quantification of microglia numbers (IBA1, cyan) in the field of view (FOV) of the frontal cortex. Original magnification: 63×; scale bar=40 μm. (C) Quantification of microglial proliferation measured by evaluating Ki67 (yellow) colocalization with IBA1⁺ (cyan) microglia in the cortex of matched sagittal sections. (D-E) Microglial morphology calculated by Sholl analysis from a total of 12 microglia from 3 matching brain sections per mouse (n=4 mice per group). (D) Representative microglia renderings and (E) Sholl analysis quantification. Original magnification: 63×; scale bar=10 μm. Statistical significance between experimental groups was calculated by one-way ANOVA with Tukey's post hoc tests from two independent experiments. Error bars represent mean±S.E.M. and each data point represents an individual mouse (B-C) or represents an average of 4 mice per group (E). Data was collected from 6 fields of view (FOV) from a total of 3 matched sagittal sections (B-C).

FIGS. S4A-S4E. Card9 deficiency does not significantly affect microglial ROS production, lipid droplet accumulation, or expression of inflammatory cytokines associated with microglial activation in 5×FAD mice. (A-D) CD11bhiCD45int cells were gated from the brains of 5-month-old 5×FAD and Card9′-5×FAD mice and probed for reactive oxygen species (ROS) production and lipid droplet accumulation in microglia by flow cytometry. (A-B) Representative flow-cytometry histograms and mean fluorescence intensity (MFI) quantification of BODIPY labeled lipid droplets in sorted CD11bhiCD45int cells. (C-D) Representative flow-cytometry histograms and MFI quantification of CellROX labeled ROS in sorted CD11bhiCD45int cells. (E) RNA was extracted from brains of 5-month-old 5×FAD, Card9^{+/-}5×FAD, and Card9^{-/-}5×FAD mice to assess inflammation. qPCR was run to evaluate expression of cytokines. Statistical significance between experimental groups was calculated by unpaired two-tailed Student's rtest (B, D) and one-way ANOVA with Tukey's post hoc tests (E). Error bars represent mean±S.E.M. and each data point represents an individual mouse.

FIGS. SSA-S5B. Card9 deficiency in the absence of Aβ does not appreciably affected microglial transcription. (A-B) RNA-Seq was performed on microglia from 5-month-old Card9^{+/+} (denoted as WT) and Card9^{-/-} mice sorted from single-cell brain suspensions using anti-CD11b⁺-coated magnetic beads and magnetic column sorting. (A) Volcano plot comparing significantly differentially expressed genes (FDR<0.1) between Card9^{-/-} and WT microglia, where 4 genes are downregulated, and 4 genes are upregulated. (B) Heatmap representation of the top overall 10 upregulated and downregulated genes between Card9′ and WT microglia.

### DESCRIPTION OF THE INVENTION

### Abbreviations

Aβ, amyloid beta; AD, Alzheimer's disease; BMDM, bone marrow-derived macrophage; CLEC, C-type lectin; CNS, central nervous system; DAM, disease-associated microglia; FDR, false discovery rate; GWAS, genome-wide association studies; intraperitoneal, i.p.; ITAM, immunoreceptor tyrosine-based activation motif; ITIM, immunoreceptor tyrosine-based inhibition motif; LDAM, lipid-droplet-accumulating microglia; MWM, morris water maze; NFT, neurofibrillary tangle; PC, principal component; RNA-seq, RNA-sequencing; ROS, reactive oxygen species; ThioS, Thioflavin S: WT, wild type.

### Definitions

Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items. “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

“Plurality” means at least two.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The terms “treating.” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

A “subject” or “patient” is a vertebrate, including a mammal, such as a human. Mammals include, but are not limited to, humans, farm animals, sport animals and pets.

The term “biological sample” refers to a sample obtained from an organism (e.g., a human patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, semen, bone marrow, circulating tumor cells, circulating DNA, circulating exosomes, and tissue or fine needle biopsy samples, urine, peritoneal fluid, aqueous humor, and pleural fluid, or cells therefrom. Biological samples may also include sections of tissues such as frozen sections or formalin fixed paraffin embedded sections akin for histological purposes. A biological sample may also be referred to as a “patient sample.”

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “compound,” as used herein, refers to any type of substance or agent that is commonly considered a drug, or a candidate for use as a drug, as well as combinations and mixtures of the above.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, phosphorodithioate, methylphosphonate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is 5′-end; the left-hand direction of a double-stranded polynucleotide sequence is referred to as 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” The term “nucleic acid construct.” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T. G. C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The term “gene” refers to a nucleic acid sequence that comprises control and coding sequences necessary for producing a polypeptide or precursor. The polypeptide may be encoded by a full-length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence, or it may include one or more introns, bound by the appropriate splice junctions.

The term “gene expression” refers to the process by which a nucleic acid sequence undergoes successful transcription and/or translation such that detectable levels of the nucleotide sequence are expressed.

The terms “gene expression profile” or “gene signature” refer to a group of genes expressed by a particular cell or tissue type wherein presence of the genes taken together or the differential expression of such genes, is indicative/predictive of a certain condition.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene in diseased tissues or cells versus normal adjacent tissue. For example, a differentially expressed gene may have its expression activated or partially or completely inactivated in normal versus disease conditions or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions but is not detectable in both. Stated another way, a gene is differentially expressed when expression of the gene occurs at a higher or lower level in the diseased tissues or cells of a patient relative to the level of its expression in the normal (disease-free) tissues or cells of the patient and/or control tissues or cells.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.A host cell that comprises a recombinant polynucleotide is referred to as a “recombinant host cell.” A gene which is expressed in a recombinant host cell wherein the gene comprises a recombinant polynucleotide, produces a “recombinant polypeptide.” A “recombinant polypeptide” or protein is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The term “amino acid” is used interchangeably with “amino acid residue,” and may refer to a free amino acid and to an amino acid residue of a peptide/protgien. It will be apparent from the context in which the term is used whether it refers to a free amino acid or a residue of a peptide/protein.

The expression “amino acid” as used herein is meant to include both natural and synthetic amino acids, and both D and L amino acids. “Standard amino acid” means any of the twenty standard L-amino acids commonly found in naturally occurring peptides/proteins. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides/proteins of the present invention, and particularly at the carboxy- or amino-terminus, can be modified by methylation, amidation, acetylation or substitution with other chemical groups which can change the peptide's/proteins's circulating half-life without adversely affecting their activity (e.g., peptidomimetic for making peptides protease resistant).

Amino acids have the following general structure:

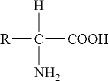

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

As used herein, amino acids are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “basic” or “positively charged” amino acid as used herein, refers to amino acids in which the R groups have a net positive charge at pH 7.0, and include, but are not limited to, the standard amino acids lysine, arginine, and histidine.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp.

The nomenclature used to describe the peptide/protein compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

As used herein, “protecting group” with respect to a terminal amino group refers to a terminal amino group of a peptide/protein, which terminal amino group is coupled with any of various amino terminal protecting groups traditionally employed in peptide synthesis. Such protecting groups include, for example, acyl protecting groups such as tert-butyl, formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS' and 3′TATGGCS' share 50% homology. As used herein. “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990. J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, a “substantially homologous amino acid sequences” includes those amino acid sequences which have at least about 95% homology, at least about 96% homology, at least about 97% homology, at least about 98% homology, or at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence, e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 96%, 97%, 98%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2X standard saline citrate (SSC), 0.1% SDS at 50° C.; including in 7% (SDS), 0.5 M NaPO4, 1 mM EDTA at 50° C. with washing in 1×SSC, 0.1% SDS at 50° C.; including 7% SDS, 0.5 M NaPO4, 1 mM EDTA at 50° C. with washing in 0.5×SSC, 0.1% SDS at 50° C.; and including in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

As used herein, an “instructional material” includes a publication, a recording, a diagram, or any other medium of expression which can be used to communicate the usefulness of the peptide/protein of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

### CARD9 Attenuates Ab Pathology and Modifies Microglial Responses in an Alzheimer's Disease

Recent advances have highlighted the importance of several innate immune receptors expressed by microglia in Alzheimer's disease (AD). In particular, mounting evidence from AD patients and experimental models indicates roles for TREM2, CD33, and CD22 in neurodegenerative disease progression. While there is growing interest in targeting these microglial receptors to treat AD, we still lack knowledge of the downstream signaling molecules used by these receptors to orchestrate immune responses in AD. Notably, TREM2, CD33, and CD22 have been described to rely on signaling through the intracellular adaptor molecule CARD9 to mount downstream immune responses outside of the brain. However, the role of CARD9 in AD was unknown. Here, it is shown that genetic ablation of CARD9 in the 5×FAD mouse model of AD results in exacerbated amyloid beta (Aβ) deposition, increased neuronal loss, worsened cognitive deficits, and alterations in microglial responses. It is further shown that pharmacological activation of CARD9 promotes improved clearance of Aβ deposits from the brains of 5×FAD mice. These results help to establish CARD9 as a key intracellular innate immune signaling molecule that regulates Aβ-mediated disease and microglial responses. Moreover, these findings suggest that targeting CARD9 might offer a novel strategy to improve Aβ clearance in AD.

### CARD9

Caspase recruitment domain-containing protein 9 is an adaptor protein of the CARD-CC protein family, which in humans is encoded by the CARD9 gene. It mediates signals from pattern recognition receptors to activate pro-inflammatory and anti-inflammatory cytokines, regulating inflammation. Homozygous mutations in CARD9 are associated with defective innate immunity against yeasts, like Candida and dermatophytes.

An example of a human CARD9 protein sequence is as follows:

(SEQ ID NO: 1)
MSDYENDDECWSVLEGFRVTLTSVIDPSRITPYLRQCKVLNPDDE
EQVLSDPNLVIRKRKVGVLLDILQRTGHKGYVAFLESLELYYPQL
YKKVTGKEPARVFSMIIDASGESGLTQLLMTEVMKLQKKVQDLTA
LLSSKDDFIKELRVKDSLLRKHQERVQRLKEECEAGSRELKRCKE
ENYDLAMRLAHQSEEKGAALMRNRDLQLEIDQLKHSLMKAEDDCK
VERKHTLKLRHAMEQRPSQELLWELQQEKALLQARVQELEASVQE
GKLDRSSPYIQVLEEDWRQALRDHQEQANTIFSLRKDLRQGEARR
LRCMEEKEMFELQCLALRKDSKMYKDRIEAILLQMEEVAIERDQA
IATREELHAQHARGLQEKDALRKQVRELGEKADELQLQVFQCEAQ
LLAVEGRLRRQQLETLVLSSDLEDGSPRRSQELSLPQDLEDTQLS
DKGCLAGGGSPKQPFAALHQEQVLRNPHDAGPAGLPGIGAVC

An example of a human nucleic acid coding for CARD9 is as follows:

| (SEQ ID NO: 2) | |
|---|---|
| 1 | aagcagaacc catcaggaag tgcacaggcg tccggcgtgc tcctccctcc ctgcagcccc |
| 61 | gggcagcatc tcccagaggc tccgcggccc aggctcctgg tgtgtctgca gtgcaggtgg |
| 121 | ctcctggaag accctcagcc tgcctgctga ggccatgtcg gactacgaga acgatgacga |
| 181 | gtgctggagc gtcctggagg gcttccgggt gacgctcacc tcggtcatcg acccctcacg |
| 241 | catcacacct tacctgcggc agtgcaaggt cctgaacccc gatgatgagg agcaggtgct |
| 301 | cagcgacccc aacctggtca tccgcaaacg gaaagtgggt gtgctcctgg acatcctgca |
| 361 | gcggaccggc cacaagggct acgtggcctt cctcgagagc ctggagctct actacccgca |
| 421 | gctgtacaag aaggtcacag gcaaggagcc ggcccgcgtc ttctccatga tcatcgacgc |
| 481 | gtccggggag tcaggcctga ctcagctgct gatgactgag gtcatgaagc tgcagaagaa |
| 541 | ggtgcaggac ctgaccgcgc tgctgagctc caaagatgac ttcatcaagg agctgcgggt |
| 601 | gaaggacagc ctgctgcgca agcaccagga gcgtgtgcag aggctcaagg aggagtgcga |
| 661 | ggccggcagc cgcgagctca agcgctgcaa ggaggagaac tacgacctgg ccatgcgcct |
| 721 | ggcgcaccag agtgaggaga agggcgccgc gctcatgcgg aaccgtgacc tgcagctgga |
| 781 | gattgaccag ctcaagcaca gcctcatgaa ggccgaggac gactgcaagg tggagcgcaa |
| 841 | gcacacgctg aagctcaggc acgccatgga gcagcggccc agccaggagc tgctgtggga |
| 901 | gctgcagcag gagaaggccc tgctccaggc ccgggtgcag gagctggagg cctccgtcca |
| 961 | ggaggggaag ctggacagga gcagccccta catccaggta ctggaggagg actggcggca |
| 1021 | ggcgctgcgg gaccaccagg agcaggccaa caccatcttc tccctgcgca aggacctccg |
| 1081 | ccagggcgag gcccgacgcc tccggtgcat ggaggagaag gagatgttcg agctgcagtg |
| 1141 | cctggcacta cgtaaggact ccaagatgta caaggaccgc atcgaggcca tcctgctgca |
| 1201 | gatggaggag gtcgccattg agcgggacca ggccatagcc acgcgggagg agctgcacgc |
| 1261 | acagcacgcc cggggcctgc aggagaagga cgcgctgcgc aagcaggtgc gggagctggg |
| 1321 | cgagaaggcg gatgagctgc agctgcaggt gttccagtgt gaggcgcagc tactggccgt |
| 1381 | ggagggcagg ctcaggcggc agcagctgga gacgctcgtc ctgagctccg acctggaaga |
| 1441 | tggctcaccc aggaggtccc aggagctctc actcccccag gacctggagg acacccagct |
| 1501 | ctcagacaaa ggctgccttg ccggcggggg gagcccgaaa cagccctttg cagctctgca |
| 1561 | ccaggagcag gttttgcgga acccccatga cgcaggccca gccggactgc cgggcattgg |
| 1621 | ggccgtttgt taagcggcac tcattttgcg gaggccatgc gggtgctcac cacccccatg |
| 1681 | cacacgccat ctgtgtaact tcaggatctg ttctgtttca ccatgtaaca cacaatacat |
| 1741 | gcatgcattg tattagtgtt agaaaacaca gctgcgtaaa taaacagcac gggtgacccg |
| 1801 | ca |

### CLEC7A

C-type lectin domain family 7 member A or Dectin-1 is a protein that in humans is encoded by the CLEC7A gene. CLEC7A is a member of the C-type lectin/C-type lectin-like domain (CTL/CTLD) superfamily. The encoded glycoprotein is a small type II membrane receptor with an extracellular C-type lectin-like domain fold and a cytoplasmic domain with a partial immunoreceptor tyrosine-based activation motif. It functions as a pattern-recognition receptor for a variety of β-1.3-linked and B-1,6-linked glucans from fungi and plants (as a member of this receptor family, dectin-1 recognizes-glucans and carbohydrates found in fungal cell walls, some bacteria and plants, but may also recognize other molecules (e.g., endogenous ligand on T-cells and ligand on mycobacteria), and in this way plays a role in innate immune response. Expression is found on myeloid dendritic cells, monocytes, macrophages and B cells. Alternate transcriptional splice variants, encoding different isoforms, have been characterized.

CLECZA has been shown to recognize species of several fungal genera, including Saccharomyces, Candida, Pneumocystis, Coccidioides, Penicillium and others.

### CARD9/CLEC7A Activators

β-Glucan is a polymer of d-glucose molecules that are linked by linear β-glycosidic bond with side branches that differ according to its sources. β-Glucans exist as (1→3)—, (1→6)-β-glucans such as zymosan, laminarin, lentinan (a β-1, 3-glucan with two β-1, 6-glucose branches every five glucose residues from Lentinus edodes), pustulan, and pleuran. They have been extracted from mushroom, barley, cereals, and seaweeds in addition to bacterial and fungal cell wall.

β(1,3)-Glucans are glucose polymers mainly linked by β-1,3-glycosidic bonds. β(1,3)-Glucans are often branched, in which side chains are attached to the backbone through 1,6-linkage. β(1,3)-Glucans typically display a triple helical structure and dissolve in alkaline solutions.

(1,6)-β-Glucans are glucose polymers having β-1,6-glycosidic bonds.

Pustulan (beta glucan Lasallia pustulata (beta 1-6-glucan) from is a median molecular weight (20 kDa), linear (1-6) linked β-D-glucan from lichen Lasallia pustulata. Pustulan is recognized by the membrane bound Dectin-1, a C-type lectin-like pattern recognition receptor. Detection of β-glucans by Dectin-1 receptor leads to the CARD9-dependent activation of NF-κB and MAP kinases. Studies have shown that pustulan can stimulate innate immune responses, inducing heat shock protein expression, eliciting phagocytosis, and production of proinflammatory cytokines.

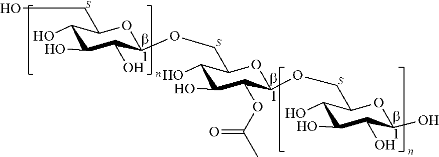

Further, one can increase expression of CARD9, such as by a genetic approach to increase CARD9 expression. For instance, a viral vector (e.g., AAV) delivering CARD9 gene expression to microglia of a subject. For example, a CARD9 gene expression cassette can be placed into a viral vector that targets expression of CARD9 in microglia. One embodiment provides an adeno-associated viral (AAV) (e.g., AAV2, AAV9 or AAV-PHP.B vectors) containing a microglia-specific promoter (e.g., Ibal, Tmem119. P2RY12, or MHCII) along with a nucleic acid sequence that codes for CARD9, such as a cassette that expresses the CARD9 gene or coding sequence.

### EXAMPLE

The following example is provided to better illustrate the claimed invention and is not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### INTRODUCTION

Alzheimer's disease (AD) is a neurogenerative disorder characterized by amyloid beta (Aβ) accumulation, the seeding of neurofibrillary tangles (NFTs), and neuroinflammation (1, 2). The culmination of these pathologies leads to neuronal loss and memory decline in patients (3). Microglia, the resident immune cells of the brain, have been heavily implicated in AD pathogenesis. More specifically, genome wide association studies (GWAS) have identified that a large percentage of late-onset AD risk loci affect genes involved in microglial biology and function (e.g., TREM2 and CD33) (4). Recent findings from AD mouse models have further solidified pivotal roles for microglia in Alzheimer's-related disease pathogenesis (5-11). It is currently thought that microglia affect AD progression through various mechanisms that include cytokine production, phagocytosis of Aβ and other neurotoxic agents, and compaction/containment of Aβ plaques. While these studies clearly define microglia as important players in AD, we are only just beginning to appreciate the molecular and effector mechanisms that microglia employ to influence neurodegenerative disease. For instance, despite ample understanding of which microglial receptors are involved in modulating AD progression, much of the intracellular signaling employed by microglia in the AD brain environment remains poorly described.

Notably, both human genetics studies and work in AD animal models have begun to uncover pivotal roles for a number of ITAM (immunoreceptor tyrosine-based activation motif)-and ITIM (immunoreceptor tyrosine-based inhibition motif)-containing receptors in AD pathogenesis. For example, emerging evidence indicates roles for the ITAM-containing receptor TREM2, as well as the ITIM-containing receptors CD33 and CD22 in AD (12-35). In the context of peripheral infection models, engagement of ITAM-containing receptors has been shown to promote the activation of CARD9 signaling and subsequent upregulation of cytokine production (36-38). In contrast, stimulation of ITIM-containing receptors provokes SHP-1 activation which potently inhibits downstream CARD9 signaling and consequently results in the dampening of cytokine production and phagocytosis (36, 37). While increasing evidence indicates that ITAM/ITIM-containing immune receptors (i.e., TREM2, CD33, and CD22) are involved in AD progression, we still lack in-depth knowledge of the intracellular signaling molecules employed by these immune receptors to influence neurodegenerative disease. CARD9 has been shown to control immune responses downstream of TREM2, CD33, and CD22 in other models of disease (36-38), although the involvement of CARD9 in AD and most other neurodegenerative disorders remains to be determined.

To explore a role for CARD9 in Alzheimer's-related disease, CARD9-deficient mice were crossed with 5×FAD mice, a well-described mouse model of Aβ-mediated neurological disease. Here it was found that genetic ablation of Card9 in 5×FAD mice leads to increased Aβ accumulation in the brain, exacerbated neuronal loss, and worsened memory decline compared with 5×FAD littermate controls. Given that CARD9 is nearly exclusively expressed by microglia in the central nervous system (CNS) (39), microglia in Card9^{-/-}5×FAD mice displayed dramatically increased proliferation while they simultaneously exhibited impaired morphological activation in response to Aβ plaques. It was further shonw that treating 5×FAD mice with a potent exogenous trigger of CARD9 signaling leads to improved control of Aβ in the hippocampus. Taken together, these findings show that the innate immune signaling molecule CARD9 is a novel regulator of Aβ-mediated neurological disease and demonstrates that targeting CARD9 activation offers a therapeutic strategy to promote Aβ clearance.

### Materials and Methods

### Mice.

All mouse experiments were conducted in accordance with the relevant regulations and guidelines of the University of Virginia (UVa) and approved by the UVA Animal Care and Use Committee (ACUC). Female 5×FAD mice (Stock #34848-JAX), Card9^{-/-} mice (Stock #017309), and C57BL/6J mice (Stock #000664) were obtained from The Jackson Laboratory and were crossed to generate Card^{+/+} (denoted as WT), Card^{+/+}, Card9%, Card^{+/+}5×FAD (denoted as 5×FAD), Card^{+/-}5×FAD, and Card9^{-/-}5×FAD experimental mice. Mice underwent a 12-hour light/dark cycle, were housed in a specific pathogen-free vivarium and at a standardized humidity (50±10%) and temperature (21±1.5° C.).

### Brain Tissue Harvest.

Experimental mice underwent euthanasia via CO₂ asphyxiation and were then transcardially perfused with ice-cold PBS. Brains were carefully harvested and bisected. The left hemisphere was then drop-fixed in 4% paraformaldehyde for 12 hours at 4° C. The right hemisphere of the brain was flash-frozen at 80° C. and stored at −80° C. Fixed samples were placed in 30% sucrose until sunken in solution and then frozen in Tissue-Plus OCT compound (Thermo Fisher) (110). Using a cryostat (Leica), brains were sectioned at 50 μm in thickness and stored in PBS+0.05% sodium azide at 4° C. until stained for imaging. For RNA extraction and protein extraction, the flash-frozen brains were thawed on ice and mechanically homogenized in 500 μl of Tissue Protein Extraction Reagent T-PER (Thermo Fisher, 78510) containing protease inhibitor cocktail complete (Roche, 11873580001) and phosphatase inhibitor cocktail PhosSTOP (Roche, 04906845001). For RNA extraction, 50 μl of brain homogenate was diluted in 500 μl Trizol and stored at −80° C. (110). The remaining brain homogenates were then spun down at 16,000 rpm for 10 minutes and the supernatant was collected for soluble amyloid beta analysis and pellet were isolated for insoluble amyloid beta analysis by ELISA.

### Immunofluorescence Microscopy.

As previously published (110), brain sections were blocked for 1 hour at room temperature with 2% donkey serum, 1% bovine serum albumin, 0.1% triton, 0.05% tween in PBS prior to incubation with primary antibodies. The primary antibodies were then diluted in this block overnight at 4° C. Brain sections were stained with anti-Aβ (D54D2, Cell Signaling, 1:300 dilution) to label plaques. To assess neuronal cell death, sections were stained with anti-NeuN (MAβ377, Millipore Sigma, 1:500 dilution) and TUNEL (Millipore Sigma, 11684795910, according to the manufacturer's instructions). In order to analyze microglia, sections were stained with IBA1 (ab5076, Abcam, 1:300 dilution). Microglial proliferation was assessed using Ki67-EF660 (SolA15, Thermo Fisher, 1:100 dilution). Microglial iron accumulation was measured using Ferritin Heavy Chain (PA5-27500, Thermo Fisher, 1:500). Following primary antibody incubation, sections were then washed 3 times for 10 minutes at room temperature in PBS and 0.05% tween-20. After washing, the brain sections were incubated in respective donkey Alexa Fluor 488, 594, 647 anti-rabbit, -goat, -rat, -streptavidin, and-mouse (Thermo Fisher, 1:1000 dilution) for 2 hours at room temperature. Once again, brain sections were washed 3 times for 10 minutes at room temperature. Following washing, sections were stained with DAPI (1:1000) for 10 minutes at room temperature to label nuclei, or stained with ThioflavinS (Sigma-Aldrich, 2 mg/10 ml) for 8 minutes followed by three 2-minute washes with 50% ethanol at room temperature to label plaques (110). Brain sections were then transferred to PBS before being mounted to glass microscope slides with 50 μl of ProLongGold antifade reagent (P36930, Invitrogen) and coverslips. For storage, mounted brain sections were kept at 4° C. and were imaged using LAS AF software (Leica Microsystems) on a Leica TCS SP8 confocal microscope. Images were analyzed using FIJI software or Imaris software (9.5.1).

### ELISA.

To assess amyloid beta composition in the brain, Aβ was measured in brain homogenates supernatants for soluble Aβ analysis, or brain homogenate pellets that underwent guanidine extraction by incubating the pellets 1:6 in 5 M guanidine HCL/50 mM tris, pH=8.0 at room temperature for 3 hours for insoluble Aβ analysis. The guanidine-extracted samples were then diluted 1:5 in PBS containing protease inhibitor cocktail complete (11873580001, Roche) then centrifuged at 16,000 g for 20 minutes at 4° C. The brain homogenate supernatant was diluted 1:10 and the guanidine-extraction supernatant was diluted 1:200 and quantified by Amyloid beta 42 Mouse ELISA kit (KMB3441, Thermo Fisher).

Mouse behavior.

The Morris water maze (MWM) was completed on 4-month-old mice and was performed as outlined in (107). The first four days of acquisition had four 60-second trials and the fifth day (probe) had one 60-second trial in which the hidden platform was removed. Mice were gently placed in an opaque 23° C. pool filled with white paint and a hidden platform 1 cm below water level. The pool contained 4 different visual cues with varying shapes and colors and mice were placed in alternate places in the pool for each trial. Mice were given 60 seconds to find the hidden platform during each trial, however, if the mouse was unable to find the platform, they were placed on the platform for 5 seconds at the end of the trial. Tracking and scoring of all behavioral trials was accomplished using video tracking software (Noldus Ethovision XT).

Aβ oligomer preparation.

Aβ (1-42) (641-15. California peptide) was monomerized using a previously published protocol (108), using hexafluoroisopropanol (HFIP) (52517, Sigma-Aldrich). 5 mM monomeric Aβ samples were incubated for 24 hours at 4° C. in F12 media to make a 200 μM stock of oligomeric Aβ. Samples were then incubated with CypHer5E-NHS ester (PA15401, GE Healthcare) diluted in 0.1 M sodium bicarbonate for 30 minutes covered and at room temperature. Following incubation, Biospin columns (7326227, Bio-Rad) were used to quench unbound dye. CypHer5E-tagged Aβ oligomers were stored at 4° C. prior to cell culture treatment or injection (110).

In vitro phagocytosis.

As previously published (110), bone marrow-derived macrophages (BMDMs) were harvested from the hind limbs of WT and Card9″ mice. Collected bones were sprayed with 70% ethanol before being placed in IMDM (12440-053, Gibco) containing penicillin/streptomycin (P/S) (15140-122, Gibco). Using a 25-gauge needle, marrow was flushed through bones using 20 ml of IMDM containing P/S. Using an 18-gauge needle, the flushed bone marrow was triturated 5 times to make a single-cell suspension. Samples were spun down at 1500 rpm for 5 minutes at 4° C. Cell pellets were resuspended in bone marrow macrophages differentiation media (BMDM media) containing IMDM, 10% FBS, 1% non-essential amino acids, 1% P/S, and 50 ng/ml M-CSF. Cells were then plated on 150×25 mm culture dishes (430597, Thomas Scientific). Three days after plating, 5 ml of BMDM media was added to each dish. On day 6, media was aspirated from dishes and 10 ml of PBS was added to each plate and incubated for 10 minutes at 4° C. Using a scraper, BMDMs were removed from the dish and transferred to a conical tube, spun down, and resuspended in BMDM media. 2 million cells/well were pipetted in 6 well plates containing glass coverslips. The next day, BMDMs were treated with 10 μM of oligomeric Aβ tagged with CypHer5E for 24 hours. BMDM-coated glass coverslips were fixed for 10 minutes at room temperature in 4% PFA. Following fixation, coverslips were washed 3 times with cold PBS. The cells were then permeabilized using 0.25% Triton X-100 diluted in PBS for 10 minutes at room temperature. Cells were washed 3 times with cold PBS. The coverslips containing BMDMs were then blocked in 2% donkey serum, 1% bovine serum albumin, 0.1% triton, 0.05% tween in PBS for 1 hour prior to incubation with primary antibodies for CD68 (MCA1957, Bio-Rad, 1:1000 dilution) and anti-Aβ (D54D2, Cell Signaling, 1:300 dilution) diluted in blocking buffer overnight at 4° C. Cells were then washed 3 times with PBS and stained with Alexa Fluor secondary antibodies (Thermo Fisher, 1:1000 dilution) for 1 hour at room temperature. Finally, cells were washed 3 times with PBS and stained with DAPI (1:1000) for 10 minutes at room temperature before mounting the coverslips onto microscope slides to assess Aβ phagocytosis.

In vivo phagocytosis.

WT and Card9^{-/-} mice received a 1 μl injection of CypHer5E-tagged Aβ oligomers (1 mg/ml) into the right-hemisphere cortex (at ±2 mm lateral, 0 mm anterior-posterior, and −1.5 mm ventral relative to the intersection of the coronal and sagittal suture (bregma) at a rate of 200 nl/min) using a stereotaxic frame (51730U, Stoelting) and nanoliter injector (NL2010MC2T, World Precision Instruments). 48 hours post injection, mice were euthanized using CO₂ and transcardially perfused before processing the brains to be imaged to assess Aβ phagocytosis (110).

Flow cytometry.

To assess BODIPY and ROS in gated microglia, mice were euthanized using CO₂ and transcardially perfused using 20 ml of PBS. After removing the meninges and choroid plexus, the brains were processed into a single cell suspension as described in (109). Cell pellets were then resuspended in 13 ml of 37% isotonic Percoll (17-0891-01. GE Healthcare) to remove myelin. Samples were spun down with no brake and the myelin layer was removed. The remaining cell pellet was transferred to a 96 well V-bottom plate and washed with 1×PBS. The assessment of lipid droplet accumulation in cells was accomplished by staining the cells with BODIPY (D3861, Invitrogen, 1:2000) diluted in PBS at 37° C. for 10 minutes. To label reactive oxygen species, cells were stained with CellROX (C10491, Thermo Fisher, 1:500) diluted in PBS at 37° C. for 30 minutes. Following BODIPY and CellROX staining, cells were spun down 1500 rpm for 5 minutes at 4° C. and washed with FACS buffer (pH 7.4, 0.1 M PBS: 1 mM EDTA, and 1% BSA). To label microglia, cells were stained with flow antibodies for CD11b, and CD45 (eBioscience) diluted 1:200 in FACS buffer for 20 minutes at 4° C. Cells were spun down and washed with FACS buffer. Prior to running samples on the cytometer, cells were resuspended in 100 μl FACS buffer containing DAPI 1:5000. Microglia were gated as live cells (DAPI negative), single cells, and as CD11bhiCD45in cells. BODIPY and CellROX mean fluorescence intensity (MFI) was measured in these gated microglia.

qPCR.

Trizol samples containing 50 μl of half-brain homogenates were thawed on ice before adding 200 μl of chloroform (BP1145-1, Fisher Scientific). Samples were incubated at room temperature for 5 minutes and spun down at 14,000 rpm at 4° C. for 15 minutes. The top fraction was collected and incubated with equal volume of isopropanol (19516, Sigma) for 10 minutes at room temperature. The samples were spun down at 12,000 rpm 4° C. for 10 minutes, followed by 2 washes with 70% ethanol. After the last wash, ethanol was aspirated off and the pellet was left to air dry for 15 minutes before adding 100 μl of DNAse/RNAse free water. Sample quality was assessed using NanoDrop 2000 Spectrophotometer (Thermo Scientific). cDNA synthesis was achieved using Sensifast cDNA Synthesis kits (BIO-65054, Bioline). Expression levels of 116 (Mm00446190_ml), Illb (Mm00434228_ml), 1118 (Mm00434226_ml), and Gapdh (Mm99999915_g1) was determined using Sensifast Probe No-ROX kit (BIO-86005, Bioline) and the CFX384 Real-Time PCR machine (1855484, BioRad) following the manufacturing protocols.

MACS isolation of microglia for RNA-sequencing.

Euthanasia of mice was performed using CO₂ followed by transcardial perfusion using 20 ml of 1×PBS with heparin. The meninges and choroid plexus were removed from the brain before beginning the magnetic-activated cell sorting (MACS)-sorting protocol. Microglia were isolated using the methods described in (109). In brief, brains were placed in 5 ml of HBSS (with Mg and Ca) (14025092, Gibco) with papain 4U/ml (LS003126, Worthington) and 50 U/ml DNase I (10104159001, Sigma-Aldrich). Following 3 triturations of the samples using a 5 ml serological pipette over 45 minutes at 37° C., the brain homogenates were transferred to a conical tube containing a 70-μm cell strainer and topped with 20 ml of DMEM/F12 (21331020, Gibco) containing 10% FBS, 1×antibiotic-antimycotic (15240096, Thermo Fisher), and 1× GlutaMAX (35050061, Invitrogen). Strained samples were then spun down with slow brake (3 on a 0-10 scale) for 10 minutes at 300 G, resuspended in 160 μl MACS buffer (130-091-376, Miltenyi Biotec), and then incubated with 20 μl MACS CD11b (microglia) microbeads (130-093-634, Miltenyi Biotec) for 15 min at 4° C. Sorting was performed using LS columns and a QuadroMACS magnet (Miltenyi, 130-042-401 and 130-091-051) according to the product instructions. The protocol efficiency was validated using flow cytometry (>90% CD11bhiCD45int) before submitting for RNA-sequencing.

RNA-sequencing analysis.

Microglia sorted using MACS isolation were sent to Azenta Next Generation Sequencing. Using splice-aware read aligner HISAT2, FASTQ files were aligned with the UCSC mm 10 mouse genome. Quality control filtering was applied using Samtools. Next, HTSeq was used to sort reads into feature counts. DESeq2 (v1.30.0) was utilized to normalize raw counts to read depth, perform principal component analysis, and carry out differential expression analysis. The Benjamini-Hochberg procedure was used to correct p-values and limit false positives arising from multiple testing. RNA-seq analyses were performed using Seq2Pathway, fgsea, tidyverse, and dplyr software packages. Heatmaps were produced using the pheatmap R package (https://github.com/raivokolde/pheatmap), lattice (http://lattice.r-forge.r-project.org/) or ggplot2 (https://ggplot2.tidyverse.org) packages. Volcano plots were produced using the Enhanced Volcano R package (https://github.com/kevinblighe/EnhancedVolcano).

Intrahippocampal injection procedure.

5×FAD and Card9^{-/-}5×FAD mice were anesthetized with a ketamine/xylazine cocktail before receiving a bilateral hippocampal injection of 2 μl of vehicle or 2 μg pustulan into the right and left hemisphere of the hippocampus (at +2 mm lateral, -2 mm posterior, and −2 mm ventral relative to the intersection of the coronal and sagittal suture (bregma) at a rate of 200 nl/min) using a stereotaxic frame (51730U, Stoelting) and nanoliter injector (NL2010MC2T, World Precision Instruments). Seven days post injection, mice were euthanized using CO₂ and transcardially perfused before preparing brains for immunofluorescent staining to evaluate Aβ clearance in the hippocampus. Images were analyzed using FIJI software or Imaris software (9.5.1).

### Statistics.

All statistical analyses were performed using Prism software (GraphPad). Statistical tests include Student's t test (paired and unpaired), one-way ANOVA, and two-way ANOVA. P values less than 0.05 were deemed significant: * p<0.05. ** p<0.01. *** p<0.001, **** p<0.0001. All data are represented as mean±SEM.

### Results

### CARD9 Signaling Restricts Brain Amyloidosis in 5×FAD Mice

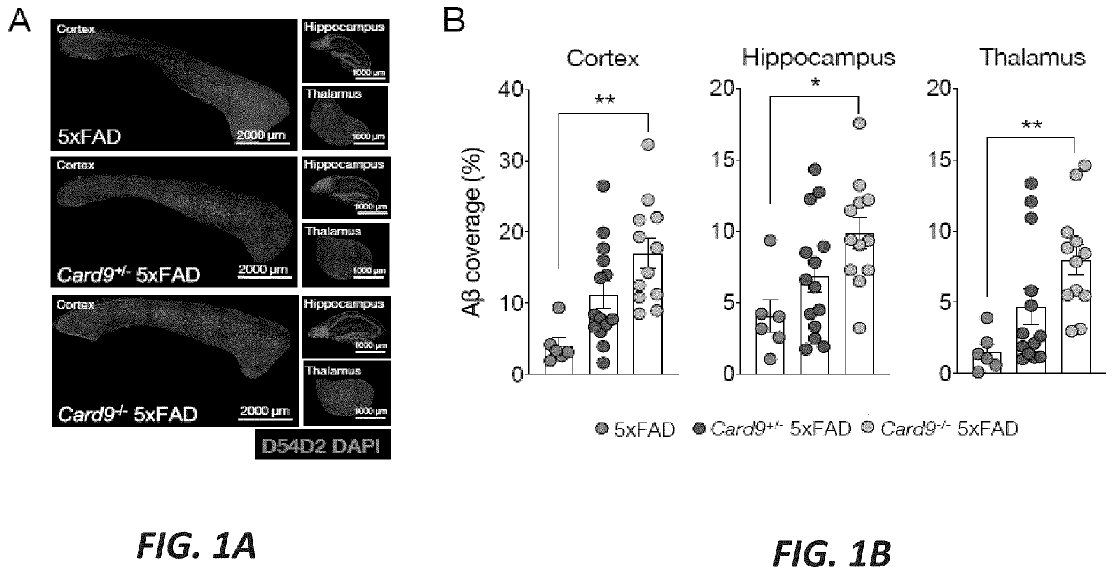

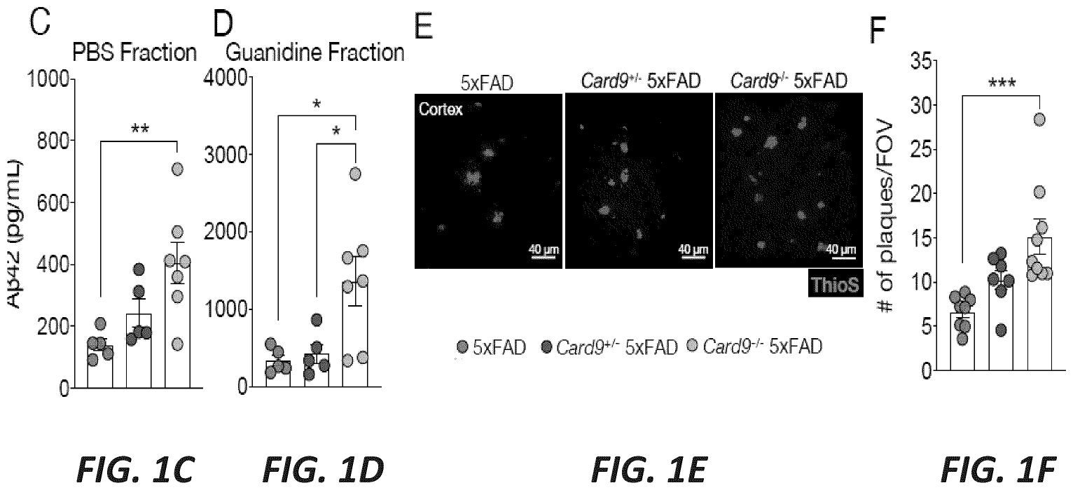

To investigate how CARD9 influences the development of Aβ pathology, a germline deletion of Card9 was introduced into 5×FAD mice, an AD mouse model characterized by early Aβ accumulation (40, 41). At 5 months of age, Card9′5×FAD mice had significantly greater Aβ burden in the cortex, hippocampus, and thalamus in comparison to Card9^{+/-}5×FAD and Card9^{+/+}5×FAD (referred to as 5×FAD mice) littermate controls (FIGS. 1A and B). Consistent with the increased levels of Aβ staining observed in 5×FAD mice that lack CARD9, an increased amounts of Aβ42, the most deleterious isoform of Aβ (42, 43), was also detected in both the soluble (PBS-extracted) and insoluble (guanidine-extracted) brain fractions obtained from Card9 1-5×FAD mice (FIGS. 1C and D). The soluble fraction of Aβ is thought to contain the most neurotoxic oligomers of Aβ (44), whereas the insoluble fraction consists of higher-order Aβ forms found in amyloid plaques (45). In alignment with the increase of Aβ in the insoluble fraction, the cortex in Card9″5×FAD also contain approximately double the number of individual plaques in comparison to 5×FAD littermate control mice (FIGS. JE and F). These data show that CARD9 plays a role in limiting Aβ accumulation in 5×FAD mice.

### The Loss of CARD9 Exacerbates Neuronal Loss and Memory Decline in 5×FAD Mice

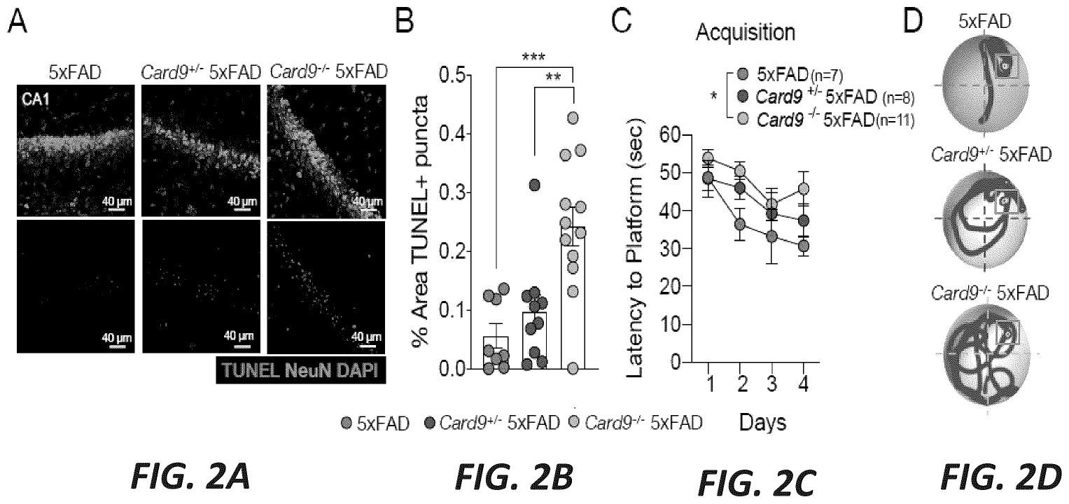

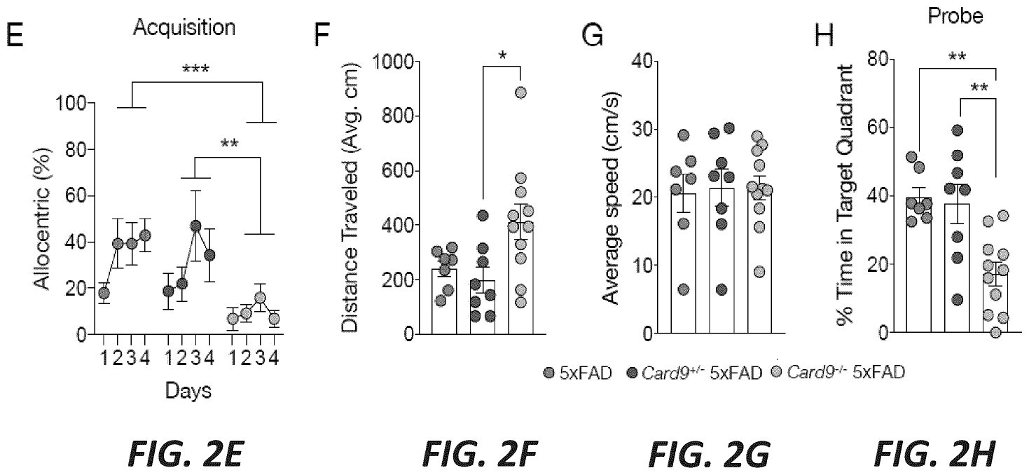

Continual accumulation of Aβ can cause appreciable neuronal damage in the AD brain (2, 46, 47) and the deleterious effects of Aβ deposition on neuronal health is thought to drive behavioral changes and pronounced learning and memory decline (48-51). To determine if the increase in Aβ accumulation seen in Card9^{-/-}5×FAD mice is accompanied by heightened neuronal loss, neuronal cell death was first evaluated by performing TUNEL staining on the CA1 region of hippocampal samples from 5×FAD, Card9^{+/+}5×FAD, and Card9^{-/-}5×FAD mice. The CA1 region of the hippocampus is densely packed with neurons forming circuits responsible for the consolidation and retrieval of memory (52). Initial seeding of Aβ often originates in the hippocampus; therefore, neuronal damage is often seen in this region, and this is thought to explain some of the learning and memory behavioral deficits commonly observed in Aβ-driven mouse models of AD (53-55). Although 5×FAD mice do not characteristically display marked neuronal loss until later stages of disease (56), 5-month-old Card9^{-/-}5×FAD mice were found to have pronounced levels of TUNEL⁺ NeuN⁺ cells in the CA1, indicative of neuronal death (FIGS. 2A and B). Taken together, these data demonstrate that impaired control of Aβ in CARD9-deficient 5×FAD mice is associated with increased levels of neuronal cell death in the CA1 region of the hippocampus.

Given the increased neuronal loss and amyloidosis observed in CARD9-deficient 5×FAD mice, it was next sought to deterkmine if Card9^{-/-5}×FAD mice also display deficits in learning and memory. To this end, the Morris water maze (MWM) behavioral test was employed to probe spatial learning and memory (57). Over the first four days of MWM, 5×FAD control mice exhibited a substantial decrease in latency to find the hidden platform which is indicative of intact spatial learning (FIG. 2C). In comparison, Card9′ 5×FAD mice spent more time searching for the hidden platform over the four acquisition days, demonstrating significantly impaired spatial learning in comparison to 5×FAD littermate controls (FIG. 2C). The navigation path used by mice to find the platform can also provide insights into the degree of cognitive dysfunction. Allocentric movement, for example, is defined by the ability of the mouse to find the hidden platform within 3 navigational turns and is dependent on hippocampal spatial memory (58). Interestingly, Card9^{-/-5}×FAD mice displayed significantly decreased hippocampal-based allocentric spatial memory when searching for the hidden platform compared with 5×FAD and Card9^{+/-}5×FAD littermate control mice (FIGS. 2D and E). Moreover, Card9^{-/-}5×FAD mice also covered a significantly longer distance in search of the hidden platform on the fourth day of the MWM test (FIG. 2F), suggesting a less targeted platform search in comparison to 5×FAD controls. Locomotor deficits did not contribute to any differences seen between groups in the MWM acquisition days, as comparable speeds were measured between 5×FAD, Card9^{+/-}5×FAD, and Card9^{-/-}5×FAD mice (FIG. 2G). Impaired spatial memory was also displayed by Card9^{-/-}5×FAD mice during probe day, as they spent significantly less time than 5×FAD and Card9^{+/-}5×FAD mice in the MWM pool target quadrant that had contained the hidden platform during the first four days of acquisition (FIG. 2H). CARD9 deletion alone in the absence of Aβ amyloidosis was not found to appreciably impact performance in the MWM test and comparable spatial learning and memory was observed in Card9^{+/+}, Card9^{+/-}, Card9^{-/-} mice (FIG. S1). These findings suggest that absence of CARD9 in 5×FAD mice leads to increased levels of neuronal cell death in the hippocampus and accelerated cognitive impairments.

### CARD9 Deficiency in 5×FAD Mice Results in Altered Microglial Responses

In recent years there has been ever-growing interest in the roles that microglia play in AD. This was initially sparked by results obtained in human genetics studies linking mutations in multiple microglial genes to late onset AD (4). Follow-up studies in AD mouse models have largely confirmed that microglia can influence various aspects of AD related pathology (14, 21, 22, 32, 35). Notably, mounting evidence suggests that microglia are critically involved in both the phagocytosis and compaction of Aβ, and that this subsequently helps to protect neurons from interacting with neurotoxic species of Aβ (30, 59). Therefore, given our findings demonstrating increased levels of Aβ and neuronal loss in CARD9-deficient 5×FAD mice, paired with the knowledge that Card9 is primarily expressed by microglia in the brain, we were interested in how CARD9 deletion affects the mobilization of microglial responses to Aβ-driven pathology.

The observed increase in Aβ load in the Card9^{-/-}5×FAD brain prompted the investigation of potential differences in the phagocytic capacity of macrophages in Card9-deficient mice. To begin, bone marrow-derived macrophages (BMDMs) were generated from Card9^{+/+} (referred to as WT) and Card9^{-/-} mice. WT and Card9^{-/-} BMDMs were stimulated with oligomeric Aβ tagged with CyPher5E, a pH sensitive dye. CypHer5E fluoresces when brought into the low-pH environment of the phagolysosome; consequently, an increased staining of CypHer5E signal suggests elevated phagocytosis of Aβ by BMDMs. In these studies, it was found that CARD9 deletion did not appreciably impact Aβ uptake by BMDMs (FIG. S2). To discern whether Card9-deficiency affects Aβ clearance in vivo, CypHer5E-tagged Aβ was injected into the cortex of WT and Card9+ mice and the presence of CypHer5E was compared relative to total Aβ in the injection site. Similar to the in vitro macrophage findings, substantial differences were not observed in the clearance of Aβ by IBA1⁺ cells between WT and Card9^{-/-} mice at 48 hours post intracortical injection of CypHer5E-tagged Aβ (FIG. S2). Collectively, these results show that Card9-deficiency does not appreciably affect the phagocytosis of Aβ by BMDMs or CNS-resident cells.

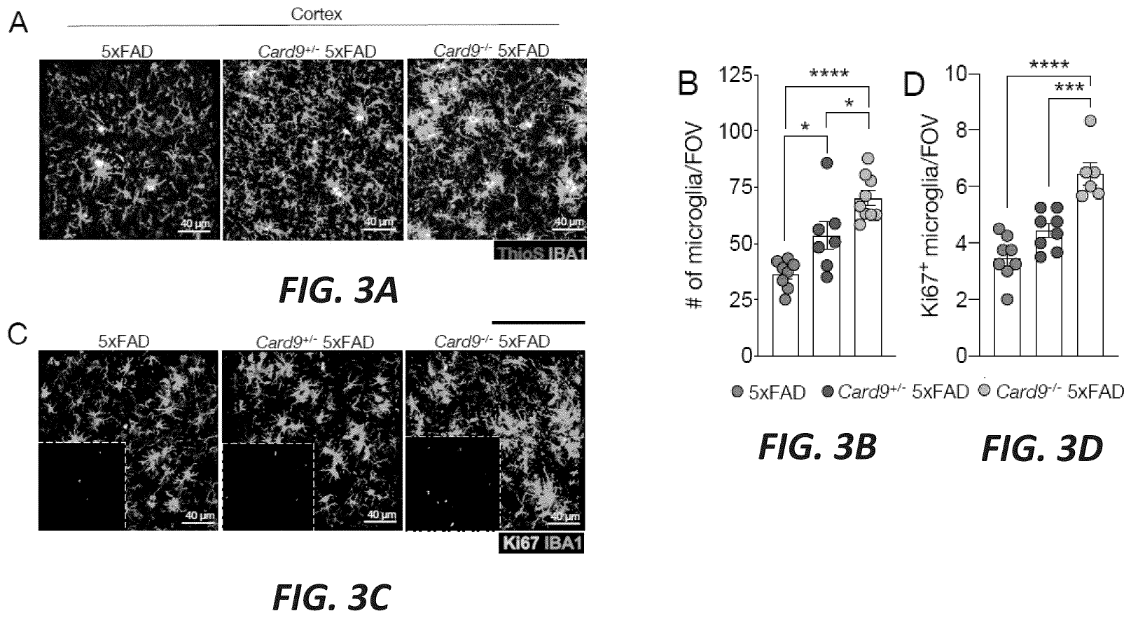

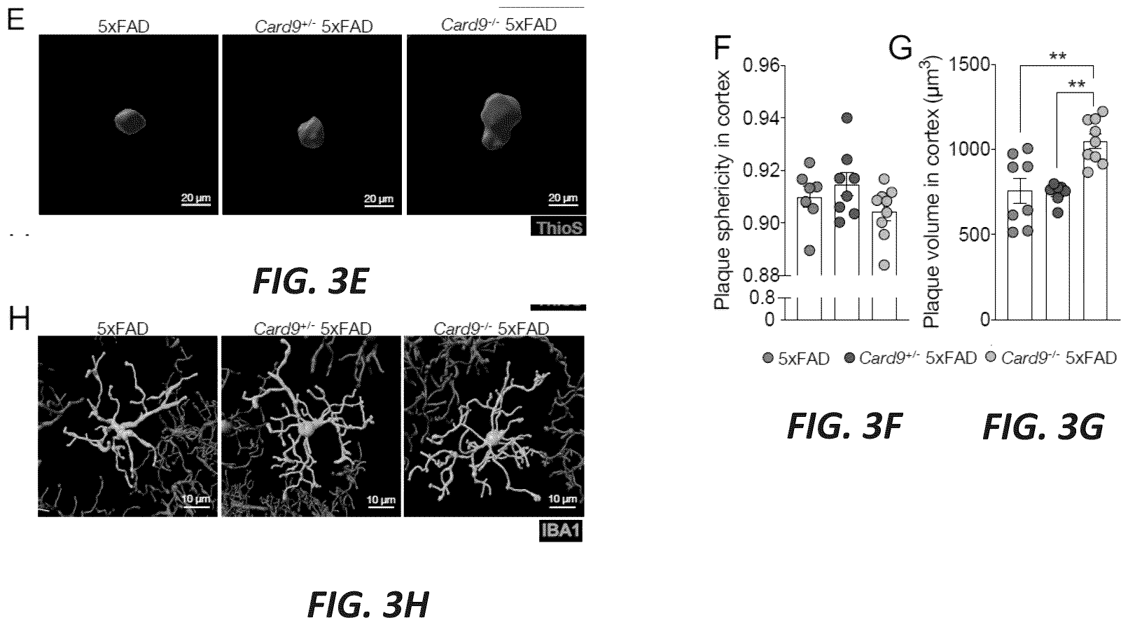

Surprisingly, it was found that the loss of Card9 results in a stepwise increase in the number of IBA1-labeled microglia within the cortex of 5×FAD mice (FIGS. 3A and B). This progressive increase in microglia between 5×FAD, Card9^{+/-}5×FAD, and Card9^{-/-}5×FAD mice is suggestive of a gene-dosage effect for the Card9 allele (FIGS. 3A and B). The approximate doubling of microglial numbers in the cortex of Card9^{-/-}5×FAD mice compared with 5×FAD controls is likely explained by a similar magnitude increase in the number of proliferating microglia (FIGS. 3C and D). More specifically, Card9^{-/-}5×FAD mice have approximately twice the number of Ki67⁺ microglia in their cortex compared with 5×FAD mice (FIGS. 3C and D). In contrast, no appreciable differences in microglial numbers or Ki67 expression were observed between CARD9-deficient and wild-type mice that lacked the 5×FAD transgenes (FIG. S3), suggesting that the increased numbers of microglia seen in Card9^{-/-}5×FAD mice was likely driven by Aβ-associated pathology.

The stimulation of microglial recruitment and proliferation in response to AD pathology is well characterized (22, 60), however, the loss of CARD9 appears to significantly enhance to microglial mobilization to Aβ plaques while concurrently exacerbating Aβ load in the cortex of 5×FAD mice. One potential explanation for these somewhat paradoxical findings of increased numbers of both microglia and Aβ load in Card9^{-/-}5×FAD mice is that the microglia recruited to Aβ in CARD9-deficient 5×FAD mice are less effective in influencing Aβ compaction. To test this, both Aβ plaque volume and sphericity were evaluated as a readout of the ability of CARD9-sufficient and -deficient microglia to shape Aβ plaque compaction (11, 22, 59). For instance, more compact plaques are believed to indicate the formation of a functional microglial barrier in which microglia physically interact with the plaque to decrease the Aβ footprint in the brain parenchyma (22, 61). While no notable differences were observed in Aβ plaque sphericity between 5×FAD and Card9^{-/-}5×FAD mice (FIGS. 3E and F), it was observed that CARD9 deficiency in 5×FAD mice leads to increased plaque volume when compared to 5×FAD littermate controls (FIGS. 3E and G). Therefore, despite the increase in microglia numbers in Card9^{-/-}5×FAD mice. Card9-deficient microglia failed to reduce the extent of Aβ plaque volume, suggesting that CARD9 is needed for microglial containment of Aβ.

It is has been extensively shown that microglia become less ramified and more ameboid in morphology in response to Aβ, and this is thought to affect the ability of microglia to handle and shape Aβ load (62). Because Card9^{-/-}5×FAD microglia exhibit increased proliferation without curbing Aβ load, it was investigated whether Card9-deficiency also impacts the ability of microglia to undergo morphological changes in response to Aβ-associated pathology. To explore this in greater detail, the Aβ-rich cortex was focused on, and progressive microglial morphological differences were identified between 5×FAD, Card9^{+/-}5×FAD, and Card9^{-/-}5×FAD mice (FIGS. 3H and I). For instance, Card9^{+/-}5×FAD and Card9^{-/-}5×FAD microglia displayed significantly more complex morphology as measured by Sholl analysis compared with 5×FAD microglia (FIGS. 3H and I). In contrast, Card9^{+/+}, Card9^{+/-}, Card9^{-/-} microglia did not exhibit morphological changes in the absence of Aβ (FIG. S3). Therefore. CARD9 is needed in driving microglial morphological activation upon Aβ stimulation. Collectively the findings suggest that CARD9 regulates microglial proliferation and morphological activation as well as Aβ compaction in the 5×FAD brain.

### Microglial ROS Production and Lipid Droplet Formation are not Impacted by CARD9 Deletion in 5×FAD Mice

While microglia can play beneficial roles in AD-related disease through their critical involvement in Aβ containment and disposal, unchecked activation of microglial inflammatory responses can also have deleterious effects and perpetuate further AD pathogenesis (8, 46). To gain a deeper understanding of how CARD9 may impact microglial-induced inflammation, it was investigated whether Card9^{-/-}5×FAD microglia take on lipid-droplet-accumulating microglia (LDAM) properties. LDAMs have been previously described in the aged brain as pro-inflammatory, with increased reactive oxygen species (ROS) and cytokine production (63). To determine if CARD9-deficient microglia in the 5×FAD brain exhibit LDAM-related dysregulation, brains were harvested from 5×FAD and Card9′5×FAD mice and levels of BODIPY were measured, a dye that labels lipid-droplets, and CellROX, a dye that fluoresces when oxidized by ROS, in CD11b^{hi}CD45^{int} cells. However, flow cytometrie analysis revealed no appreciable differences in BODIPY or CellROX staining in Card9^{-/-}5×FAD microglia when compared to 5×FAD control microglia (FIG. S4). Thus, Card9-deficiency does not significantly increase the prevalence of LDAMs or ROS production by microglia in the 5×FAD brain.

Aberrant production of proinflammatory cytokine production by microglia is also believed to contribute to the propagation of AD pathology and memory deficits. For instance, the cytokines IL-6, IL-1B, and IL-18 are often elevated in AD and have been reported to provoke Aβ accumulation and cognitive decline (64-67). CARD9 is known to regulate NF-κB activation, a transcription factor that coordinates the production of several pro-inflammatory cytokines including IL-6, pro-IL-1B, and pro-IL-18 (68). Therefore, the levels of key pro-and anti-inflammatory cytokines were investigated in the whole brain of 5×FAD, Card9^{+/-}5×FAD, and Card9^{-/-}5×FAD mice. However, altered levels of IL-la, IL-1B, IL-4, IL-6, IL-10, IL-17, IFN-γ, TNF-α were not observed by multiplex ELISA (FIG. S4). Altogether, these findings suggest that CARD9 deletion in 5×FAD mice does not substantially influence proinflammatory cytokine levels, production of ROS, or lipid droplet accumulation by microglia.

### Effects of CARD9 Deletion on the Microglial Transcriptional Response in 5×FAD Mice

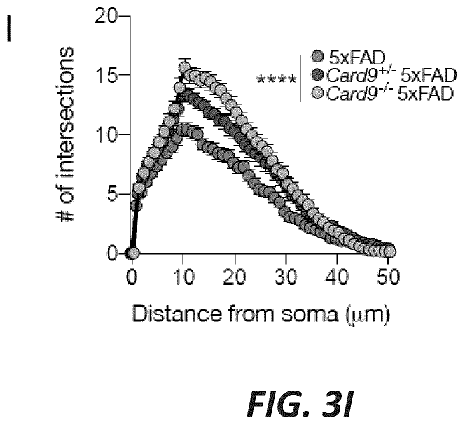

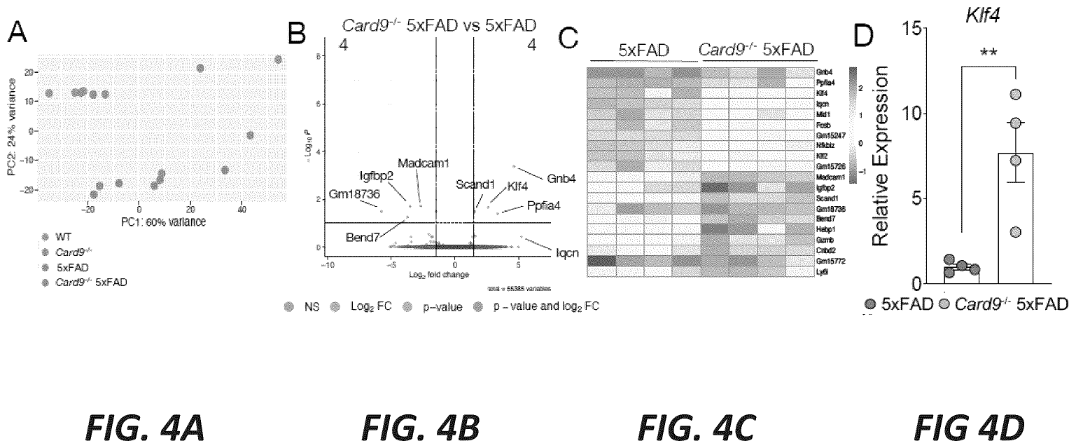

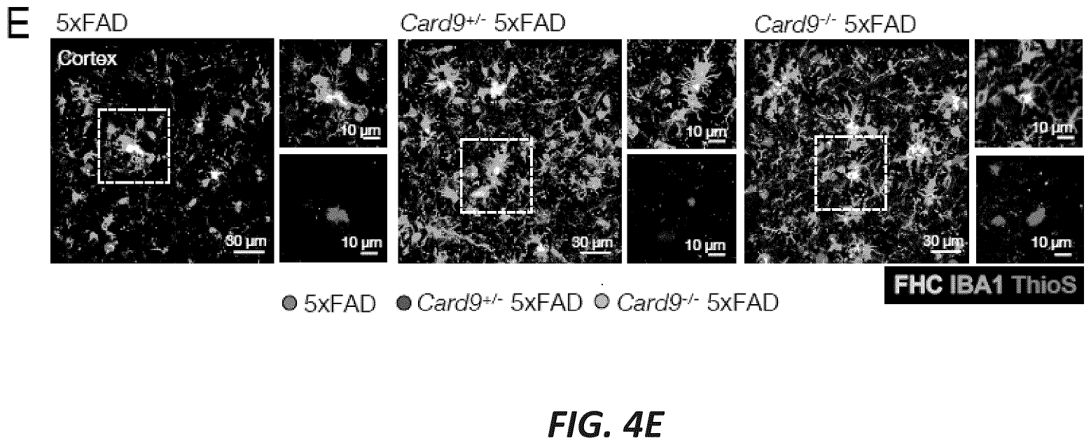

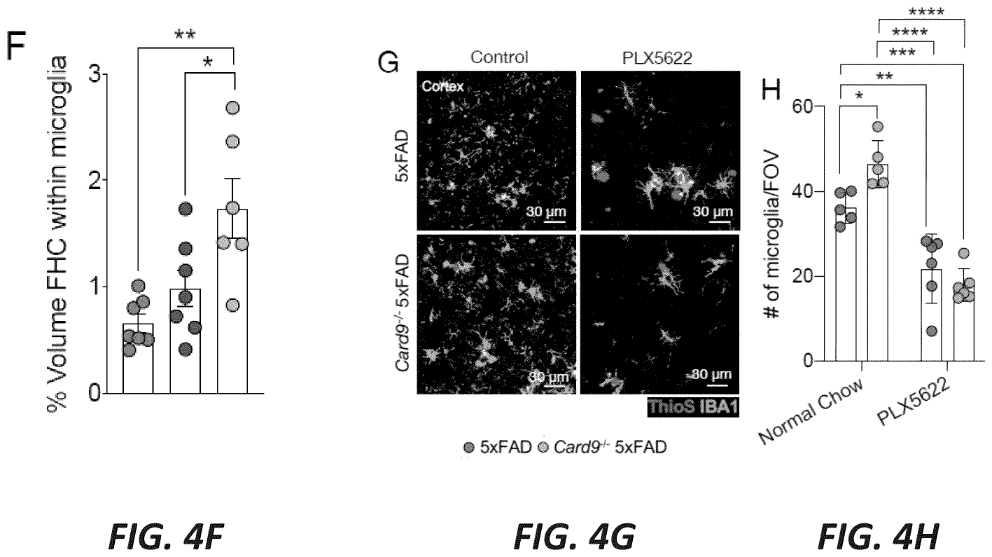

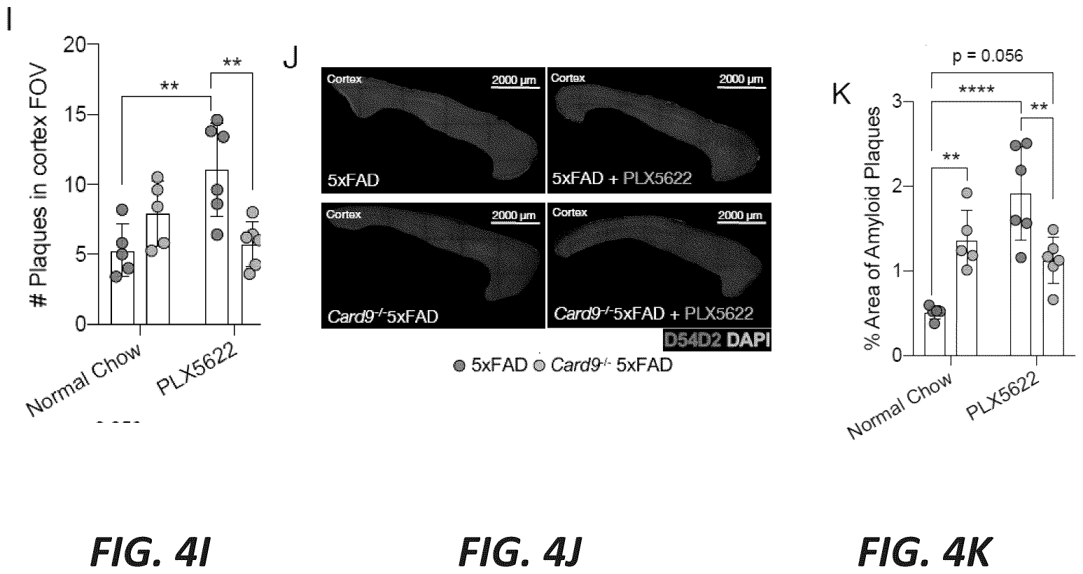

Microglia exposed to AD-associated pathology are thought to undergo a transcriptional shift to become disease associated microglia (DAMs) (69). Upon stimulation, DAMs downregulate their homeostatic markers and subsequently upregulate several activation markers in a biphasic manner (70). This process of DAM acquisition is thought to supply microglia with an increased capacity to respond to and eliminate AD pathology (71). Given that Card9-deficiency exacerbates disease progression in 5×FAD mice, it was hypothesized that the microglia of Card9′-5×FAD mice would be unable to undergo the transcriptional evolution seen in DAMs. Thus, to evaluate how CARD9^{+/+} may affect microglia activity in an unbiased and comprehensive manner, bulk RNA sequencing (RNA-Seq) was performed on magnetic bead-sorted CD11b⁺ cells from the brains of Card9^{+/+} (WT), Card9^{-/-}, 5×FAD, and Card9^{-/-}5×FAD mice. Principal component (PC) analysis uncovered a prominent separation between homeostatic (WT and Card9^{-/-}) and 5×FAD (5×FAD and Card9^{-/-}5×FAD) microglia (FIG. 4A).

Surprisingly, a major effect of CARD9 deletion was not observed on the microglial transcriptional response in 5×FAD mice (FIGS. 4B and C). In fact, when comparing Card9^{-/-}5×FAD microglia to 5×FAD microglia only 4 downregulated (Madcam1, Igfbp2, Scandl, and Gm18736) and 4 upregulated (Gnb4, Ppfia4, Klf4, and lqcn) genes (FDR<0.1) were detected (FIGS. 4B and C). However, the upregulation of Klf4 in Card9^{-/-}5×FAD microglia was observed and analyzed by RNA-Seq and qPCR (FIGS. 4C and D), which was compelling due to the fact that KLF4 has been shown to regulate microglia-driven neuroinflammation and neuronal loss in the context of AD pathology (72, 73). Although no overt changes in inflammatory signaling or oxidative stress was observed between 5×FAD and Card9^{-/-}5×FAD mice (FIG. 4 and FIG. S4) which have previously been described to be regulated by KLF4, a significant increase was observed in neuronal cell death in the CA1 region of the hippocampus (FIGS. 2A and B). Neuronal cell loss is a key element contributing to AD progression that is reported to be propagated by KLF4 (72-75). Interestingly, KLF4 is believed to contribute to microglial iron dyshomeostasis that promotes impaired microglial response to Aβ (73). In fact, iron-laden microglia have been identified in the AD brain (76). Therefore, ferritin heavy chain accumulation was looked at in microglia, a subunit of ferritin critical for iron sequestration (77). The increase in microglial coverage in the cortex of Card9″ 5×FAD mice was taken into account by normalizing the volume of ferritin heavy chain to the total volume of IBA+ cells; thus, allowing one to calculate ferritin heavy chain level per microglia. Interestingly, a striking increase in ferritin heavy chain staining was observed within Card9′5×FAD microglia compared with 5×FAD controls (FIGS. 4E and F). Thus, CARD9 likely regulates iron homeostasis in 5×FAD microglia. It is of note that Card9-deletion at steady-state in the absence of 5×FAD transgene did not cause a major transcriptional shift in microglia, with a unique 4 downregulated (Ly6i, Hebpl, Tppp3, and Cd300e) and 4 upregulated (Cacnb2, Prkcq, Ppfia4, Mrc2) genes compared with WT microglia (FDR<0.1) (FIG. S5). These findings indicate that CARD9 deletion in 5×FAD mice leads to elevated expression of KLF4 in the brain and that this is associated with increased production of ferritin heavy chain by IBA1+ cells.

### The Impact of Card9 Deletion on Aβ Load Does Not Rely on Increased Numbers of Microglia

Microglia are known to play both beneficial and detrimental roles in the context of Aβ pathology (5, 6, 21, 69, 78, 79). For instance, microglia are able to shield neurons from neurotoxic species of Aβ (11, 30); however, recent studies also suggest that microglia can contribute to the pathogenic spread of Aβ amyloidosis (6, 80). Therefore, the possibility exists that the increased microgliosis observed in Card9-deficient 5×FAD mice (FIGS. 3A and B) may contribute to the exacerbated AD pathology progression seen in Card9^{-/-}5×FAD mice (FIG. 1).

To test this, 1.5-month-old 5×FAD and Card9′-5×FAD mice were fed with either normal chow or food containing the CSFIR inhibitor PLX5622 to deplete microglia. Mice remained on PLX5622 chow or normal feed for 2.5 months and at 4 months of age the brains were collected to evaluate microgliosis and Aβ load. Similar levels of microglia depletion were observed in 5×FAD and Card9^{-/-}5×FAD mice after 2.5 months of continuous treatment with PLX5622 food (FIG. 4 G and H). PLX5622-treated 5×FAD mice were found to have significantly increased levels of Aβ plaque load in the cortex when compared with 5×FAD mice on normal chow (FIGS. 4 G, I, J, and K). In contrast, microglia depletion in Card9′-5×FAD mice did not significantly impact Aβ plaque load, as the coverage of Aβ in the cortex was commensurate between PLX5622-treated and control chow-treated Card9^{-/-}5×FAD mice (FIGS. 4 G. I, J, and K). These collective findings suggest that the increased numbers of microglia are likely not causative in the exacerbated AD pathology development of CARD9-deficient mice.

The ability of β-glucan treatment to reduce Aβ load in 5×FAD mice is dependent on CARD9

Thus far, it has been have demonstrated herein that CARD9 deletion is detrimental for microglial activation and pathology progression in the 5×FAD mouse model. These findings suggest that CARD9 activity plays a protective role in response to Aβ pathology, thus, it was sought to explore whether CARD9 stimulation can reduce Aβ load in the brain. CARD9 signaling was enhanced by targeting the activation of CLEC7A, a receptor that has been extensively reported to promote CARD9 activation in response to fungal triggers (81, 82). CLEC7A can initiate downstream CARD9 signaling following its binding to β-D-glucans, a component of yeast cell walls (83). Pustulan, a β-D-glucan that can activate a range of fungal recognition signaling pathways including CARD9, was injected into the Aβ-laden hippocampus of 5×FAD mice to evaluate how the activation of the CLEC7A-CARD9 pathway affected Aβ load.

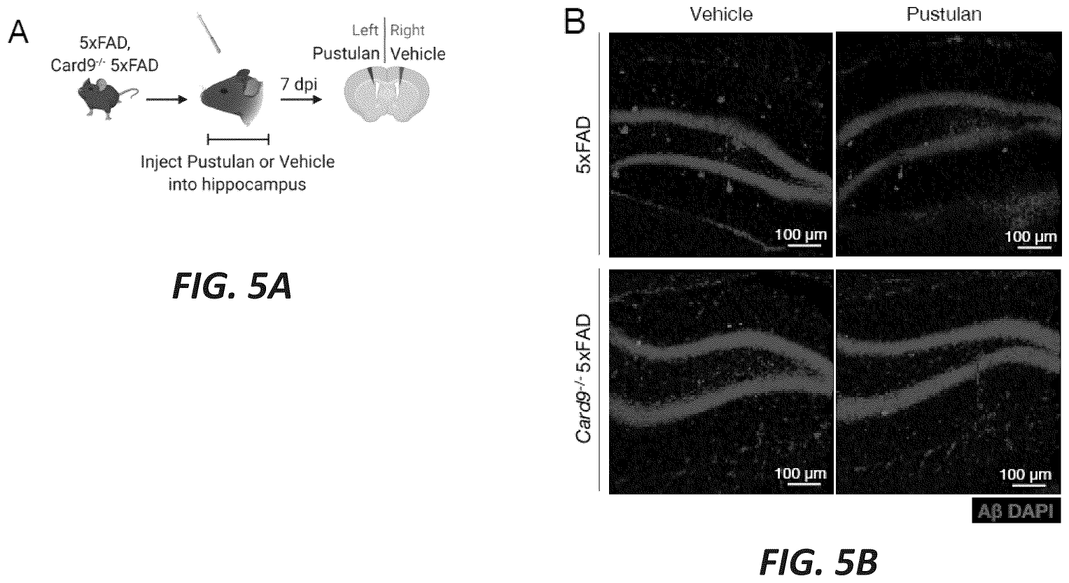

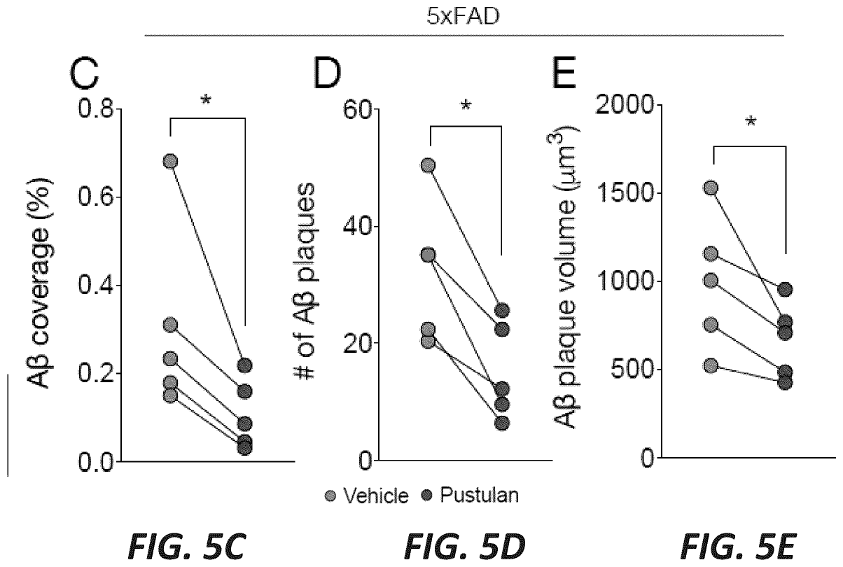

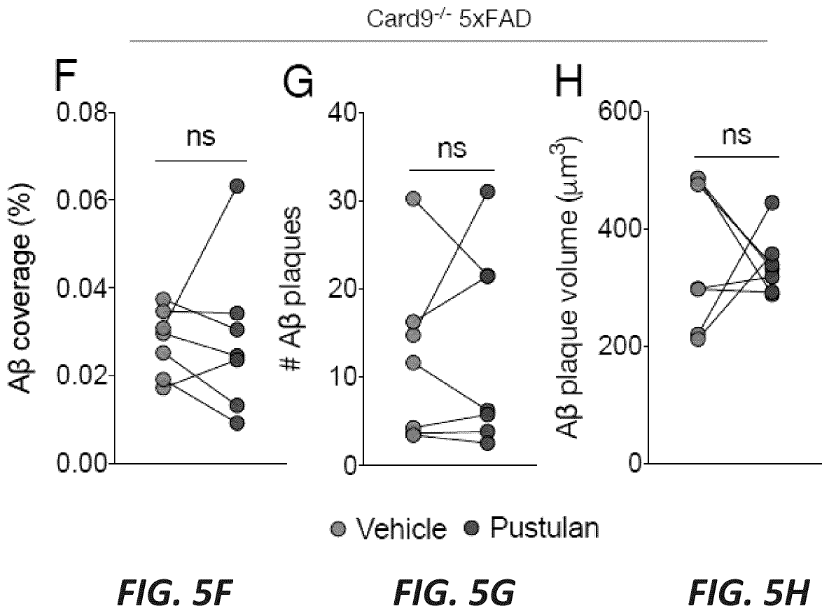

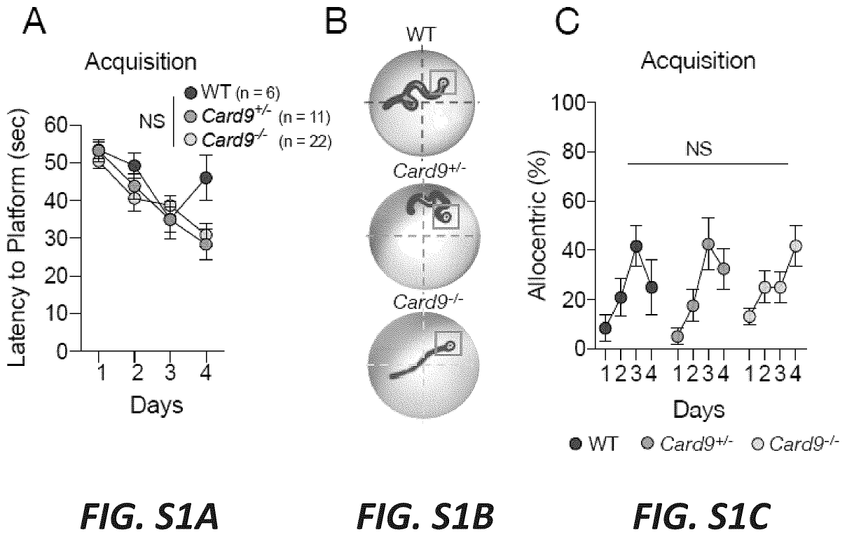

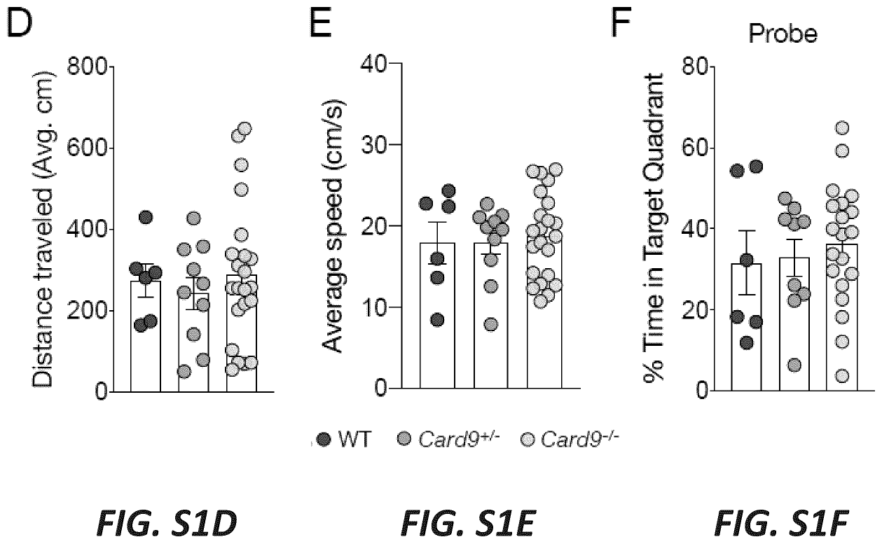

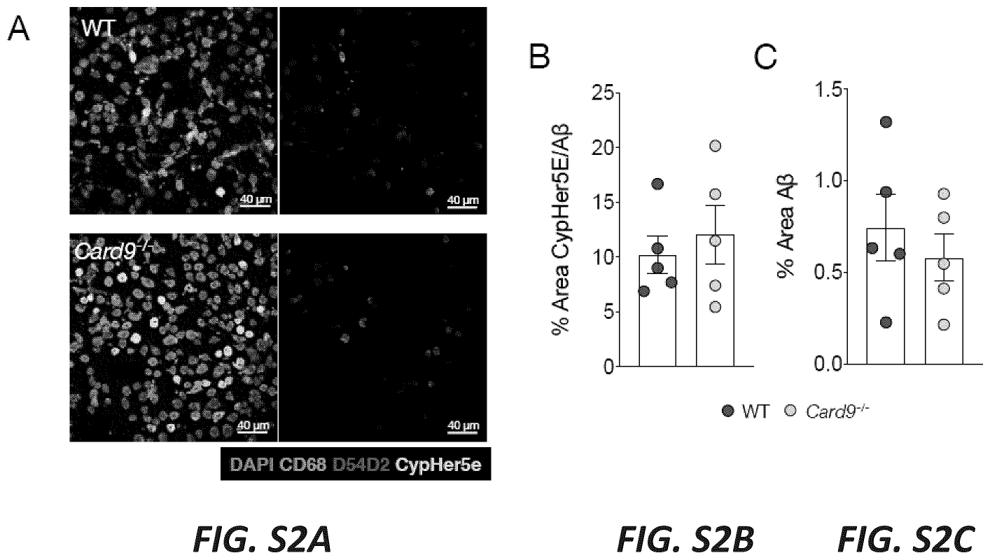

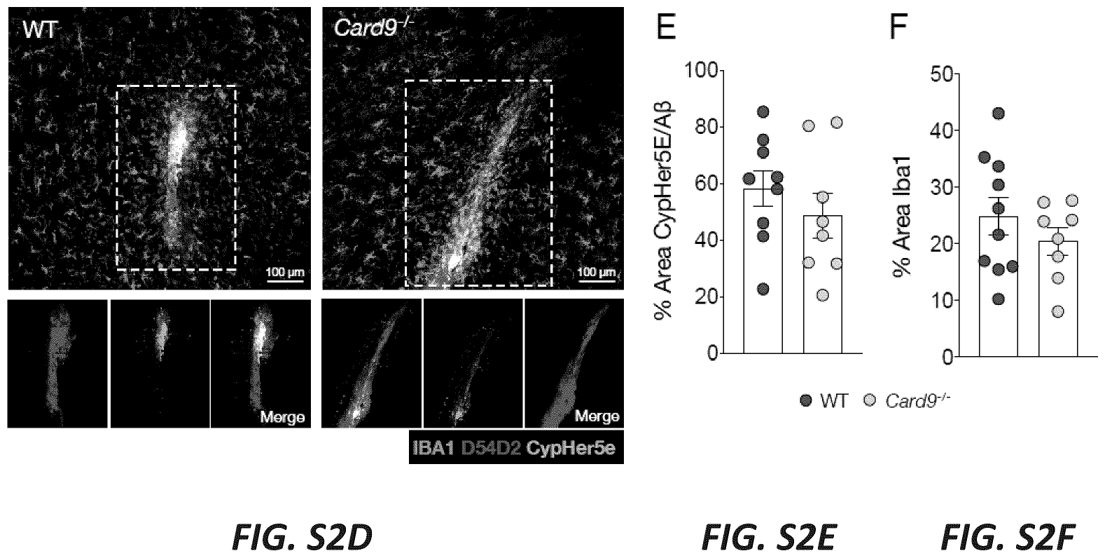

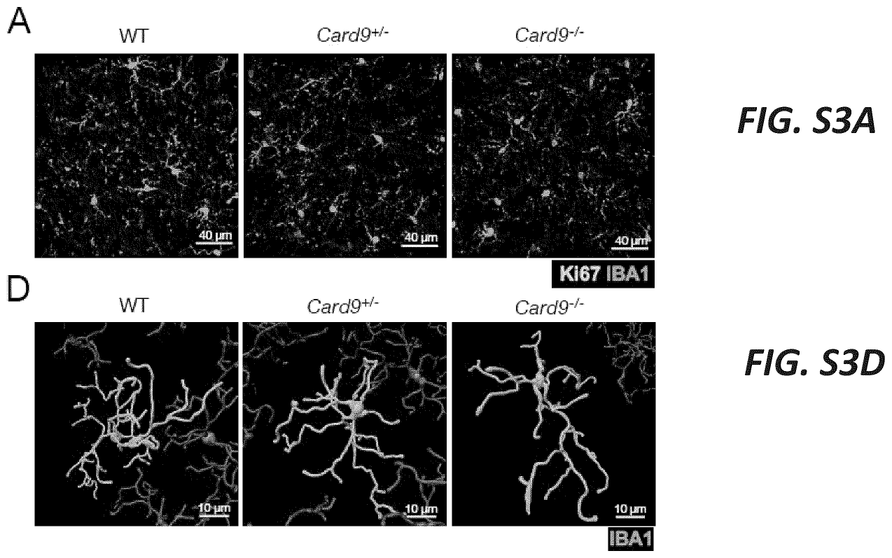

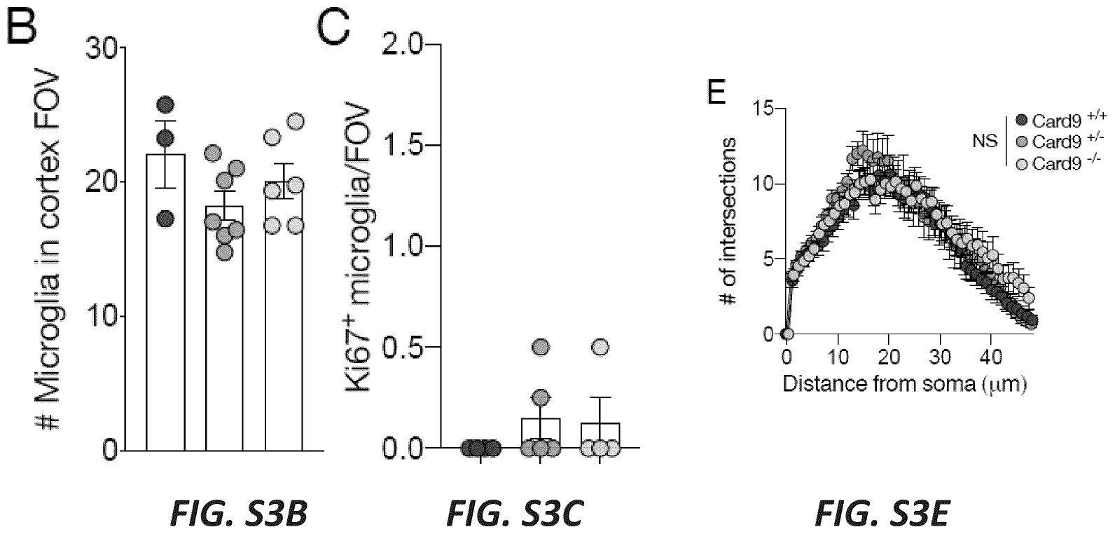

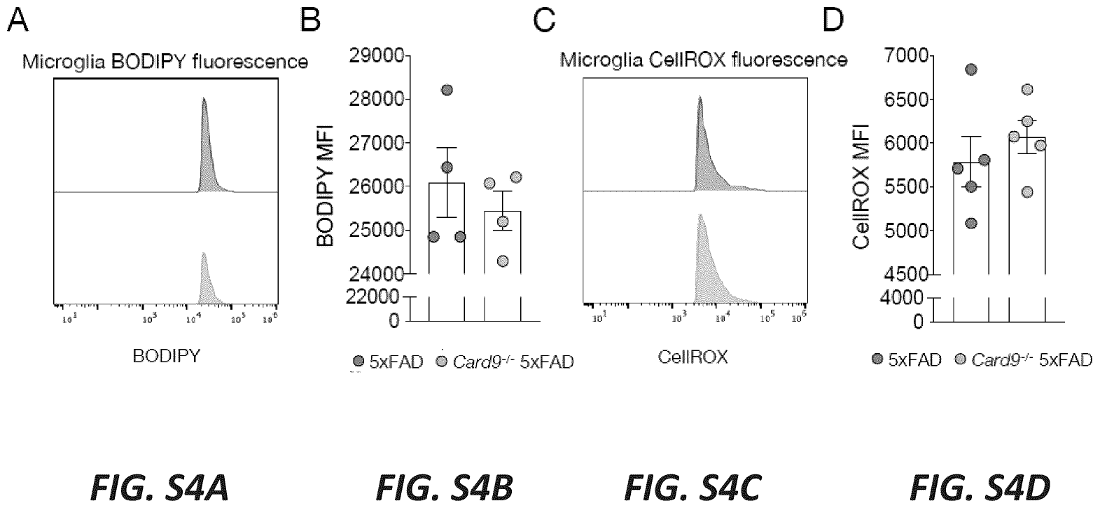

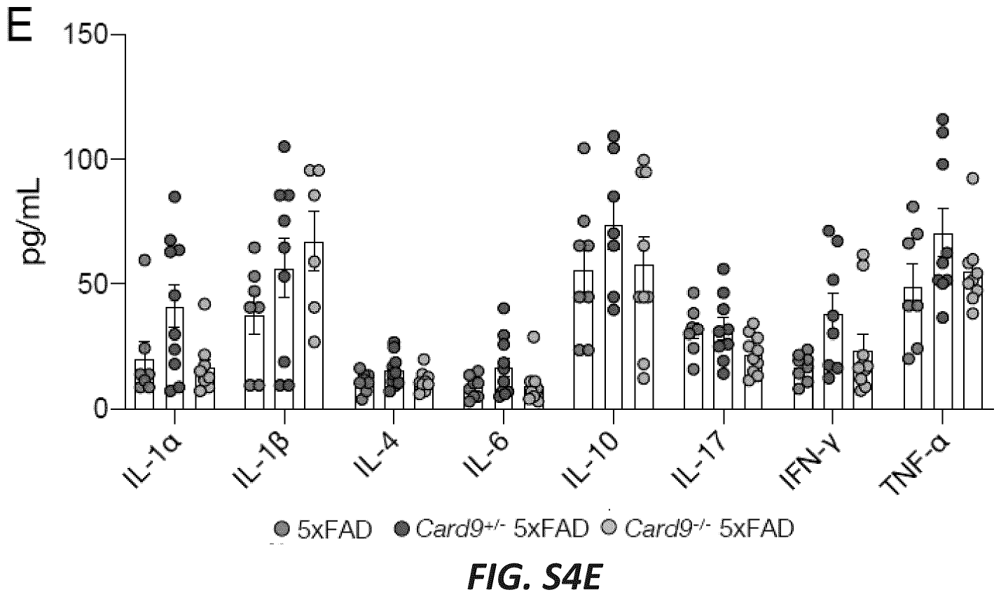

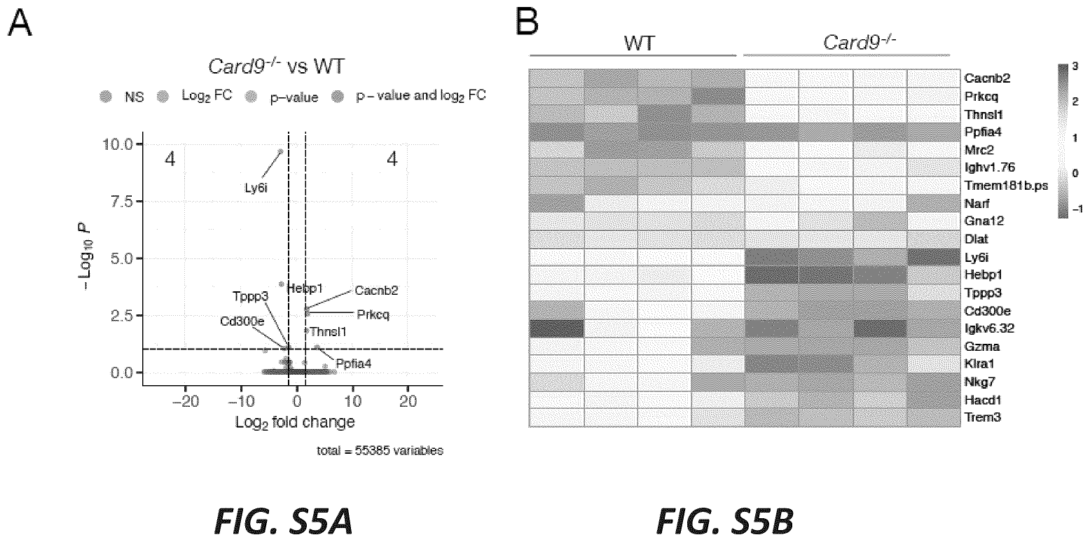

2-month-old 5×FAD and Card9^{-/-}5×FAD mice were injected with either vehicle or 2 μg of pustulan into the right and left hemisphere of the hippocampus, respectively. Card9^{-/-}5×FAD mice were included in these studies as controls to test for any potential off-target and CARD9-independent effects of pustulan treatment on Aβ pathology. Brains were then harvested at 7 days post-injection and Aβ plaque load was compared between the vehicle- and pustulan-treated hemispheres of the hippocampus (FIG. 5A). 2 months-of-age was chosen due to the appreciable Aβ deposition in the hippocampus of mice at this time point. Moreover, there are not yet statistically significant differences in Aβ load between 5×FAD mice and Card9^{-/-}5×FAD mice at this age, which enables one to more accurately assess whether pustulan has any CARD9-independent effects. In 5×FAD mice, the hippocampal hemisphere treated with pustulan had significantly reduced Aβ coverage, fewer plaques, and lower plaque volume compared to the vehicle-treated hippocampal hemisphere (FIGS. 5 B-E). In contrast, the hippocampi of Card9^{-/-}5×FAD mice had comparable levels of Aβ regardless of treatment (FIGS. 5 B and F-H), suggesting that pustulan promotes Aβ control in a CARD9-dependent manner. Taken together, these data indicate that CARD9 signaling contributes to the restriction of Aβ-driven pathology in the 5×FAD mouse model of AD.

### DISCUSSION

The identification of microglial AD risk genes from GWAS studies has implicated several receptors in both aberrant and protective microglial responses (4, 70, 84). The characterization of these microglial receptors in mice and humans has uncovered several potential therapeutic targets (85-87); the activation or inhibition of these receptors has dominated microglia-targeted AD therapy as they extensively influence microglial response to AD pathology (6, 14, 20, 22, 32, 35, 59). In particular, activation of the TREM2 receptor has been demonstrated to exert neuroprotective effects and has advanced to phase 2 clinical trials for AD treatment (85). However, much of the microglial downstream signaling in AD remains poorly defined, potentially leaving a stone unturned for additional and more precise interventions. The intricacies of microglial function in AD, and how best to target them, are more likely to be uncovered by elucidating the unique and shared downstream molecular underpinnings of these receptors.

In the studies presented here, CARD9, an immune molecule downstream of several microglial receptors implicated in AD, was identified as a regulator of microglial activation in the context of Aβ-driven pathology in 5×FAD mice. Herein it was demonstrated that CARD9 regulates the microglial response to Aβ in the 5×FAD brain, ultimately impacting Aβ load and neuronal loss. In addition, it was found that CARD9 protects against cognitive impairment in 5×FAD mice. A transcriptional comparison between 5×FAD and Card9′5×FAD microglia uncovered increased expression of Klf4 in CARD9-deficient microglia, a transcription factor linked with increased neuronal loss and iron dyshomeostasis (72, 73, 75). Interestingly, in addition to increased neuronal loss seen in the hippocampus, CARD9-deficient 5×FAD mice also display significantly higher iron retention in their microglia, a phenotype emblematic of microglial dysfunction (88, 89). Furthermore, it was demonstrated that pustulan-induced activation of CARD9 drives a reduction in Aβ plaque load in the hippocampus of 5×FAD mice. Together, the studies suggest a role for CARD9 in regulating microglial activation and Aβ load.

CARD9 has classically been defined for its roles in driving myeloid cell inflammatory responses in the context of peripheral fungal infection (68, 83, 90, 91). The ability of microglia to function in the context of infection has long been known (92-95), but whether this mirrors microglial responses to AD pathology remains ill-defined. Interestingly, artifacts of fungal pathogens adjacent to Aβ plaques have been found in the brains of AD patients (96, 97), and although it remains controversial whether fungal infection precedes or follows AD onset (98), microglia are well-equipped to recognize and respond to this pathogen in the brain (92). Thus, microglia contain sophisticated machinery that responds to both infectious triggers and AD pathology (8, 10, 94, 99); in turn, microglia have the potential to act as a critical bridge between immune activation and AD pathogenesis. For example, CLEC7A, a receptor upstream of CARD9, is upregulated by microglia surrounding fungal aggregates and Aβ plaques (35, 99). Interestingly, fungal pathogens contain an amyloid-like structure on their cell surface that allows for pathogen adhesion and biofilm formation (100, 101), which may explain the multi-modal upregulation of a fungal receptor such as CLEC7A. Altogether, the characterization of this innate immune response in both modalities of brain infection and neurodegeneration may uncover enigmatic etiologies of AD.

Targeting microglia in AD has become increasingly more complex as scientists gain a more complete understanding of the pleiotropic roles that microglia play in the evolution of disease. Just as AD can be stimulated by vastly heterogeneous triggers involving a combination of aging, environmental factors, and genetics (84, 102-105), microglial functions can also be significantly affected by these same contributing factors. Importantly, the progression of microglial transcription throughout AD pathogenesis has the capacity to significantly alter microglial responses and subsequent AD progression (33, 35, 69, 70, 106). Recent work has described the importance of these innate myeloid cells taking on a DAM signature during AD, promoting enhanced phagocytic and inflammatory function (69). However, more recent work has demonstrated that microglia undergo a phasic shift from phagocytic and proliferative to anti-viral and apoptotic in the context of tauopathy (106). In the latter phase, microglia are immunosuppressed, but also contribute to chronic inflammation, a pathogenic hallmark in the AD brain (106). Ultimately, effectively targeting microglia in AD, either through the enhancement or diminution of microglial function, may largely depend on the prevailing pathology, stage of disease, and the delincation of influential microglial signaling.

Altogether, differential microglial responses in AD, including proliferation, activation, and pathology clearance, involve critical downstream signaling that have remained poorly described to date. In the studies presented here, it has been demonstrated that CARD9 functions to impede AD pathology progression and acts as a relevant intracellular mediator of microglial response to Aβ. The findings highlight the important nature of CARD9, a shared downstream molecule of several AD-associated microglial receptors, in protecting against Aβ-mediated neurodegeneration.

### BIBLIOGRAPHY

1. M. A. DeTure, D. W. Dickson, The neuropathological diagnosis of Alzheimer's disease. Mol Neurodegener 14, 32 (2019).
2. M. T. Heneka et al., Neuroinflammation in Alzheimer's disease. Lancet Neurol 14, 388-405 (2015).
3. H. Jahn, Memory loss in Alzheimer's disease. Dialogues Clin Neurosci 15, 445-454 (2013).
4. S. D. Mhatre, C. A. Tsai, A. J. Rubin, M. L. James, K. I. Andreasson, Microglial malfunction: the third rail in the development of Alzheimer's disease. Trends Neurosci 38, 621-636 (2015).
5. E. E. Spangenberg et al., Eliminating microglia in Alzheimer's mice prevents neuronal loss without modulating amyloid-beta pathology. Brain 139, 1265-1281 (2016).
6. E. Spangenberg et al., Sustained microglial depletion with CSF1R inhibitor impairs parenchymal plaque development in an Alzheimer's disease model. Nat Commun 10, 3758 (2019).
7. E. G. Reed-Geaghan, A. L. Croxford, B. Becher, G. E. Landreth, Plaque-associated myeloid cells derive from resident microglia in an Alzheimer's disease model. J Exp Med 217 (2020).
8. H. Sarlus, M. T. Heneka, Microglia in Alzheimer's disease. J Clin Invest 127, 3240-3249 (2017).
9. D. Tejera. M. T. Heneka, Microglia in Neurodegenerative Disorders. Methods Mol Biol 2034, 57-67 (2019).
10. D. V. Hansen, J. E. Hanson, M. Sheng, Microglia in Alzheimer's disease. J Cell Biol 217, 459-472 (2018).
11. C. Condello, P. Yuan, A. Schain, J. Grutzendler, Microglia constitute a barrier that prevents neurotoxic protofibrillar Abeta42 hotspots around plaques. Nat Commun 6, 6176 (2015).
12. E. M. Bradshaw et al., CD33 Alzheimer's disease locus: altered monocyte function and amyloid biology. Nat Neurosci 16, 848-850 (2013).
13. A. Griciuc et al., Alzheimer's disease risk gene CD33 inhibits microglial uptake of amyloid beta. Neuron 78, 631-643 (2013).
14. J. V. Pluvinage et al., CD22 blockade restores homeostatic microglial phagocytosis in ageing brains. Nature 568, 187-192 (2019).
15. Y. Zhao et al., TREM2 Is a Receptor for beta-Amyloid that Mediates Microglial Function. Neuron 97, 1023-1031 e1027 (2018).
16. T. R. Jay et al., TREM2 deficiency eliminates TREM2+ inflammatory macrophages and ameliorates pathology in Alzheimer's disease mouse models. J Exp Med 212, 287-295 (2015).
17. T. R. Jay et al., Disease Progression-Dependent Effects of TREM2 Deficiency in a Mouse Model of Alzheimer's Disease. J Neurosci 37, 637-647 (2017).
18. S. M. Bemiller et al., TREM2 deficiency exacerbates tau pathology through dysregulated kinase signaling in a mouse model of tauopathy. Mol Neurodegener 12, 74 (2017).
19. P. J. Cheng-Hathaway et al., The Trem2 R47H variant confers loss-of-function-like phenotypes in Alzheimer's disease. Mol Neurodegener 13, 29 (2018).
20. T. K. Ulland et al., TREM2 Maintains Microglial Metabolic Fitness in Alzheimer's Disease. Cell 170, 649-663 e613 (2017).
21. Y. Wang et al., TREM2 lipid sensing sustains the microglial response in an Alzheimer's disease model. Cell 160, 1061-1071 (2015).
22. Y. Wang et al., TREM2-mediated early microglial response limits diffusion and toxicity of amyloid plaques. J Exp Med 213, 667-675 (2016).
23. J. D. Ulrich, T. K. Ulland, M. Colonna, D. M. Holtzman, Elucidating the Role of TREM2 in Alzheimer's Disease. Neuron 94, 237-248 (2017).
24. C. E. G. Leyns et al., TREM2 deficiency attenuates neuroinflammation and protects against neurodegeneration in a mouse model of tauopathy. Proc Natl Acad Sci USA 114, 11524-11529 (2017).
25. W. M. Song et al., Humanized TREM2 mice reveal microglia-intrinsic and -extrinsic effects of R47H polymorphism. J Exp Med 215, 745-760 (2018).
26. Y. Zhou, T. K. Ulland, M. Colonna, TREM2-Dependent Effects on Microglia in Alzheimer's Disease. Front Aging Neurosci 10, 202 (2018).
27. X. Xiang et al., The Trem2 R47H Alzheimer's risk variant impairs splicing and reduces Trem2 mRNA and protein in mice but not in humans. Mol Neurodegener 13, 49 (2018).
28. C. B. Lessard et al., High-affinity interactions and signal transduction between Abeta oligomers and TREM2. EMBO Mol Med 10 (2018).
29. C. E. G. Leyns et al., TREM2 function impedes tau seeding in neuritic plaques. Nat Neurosci 22, 1217-1222 (2019).
30. C. Condello, P. Yuan, J. Grutzendler, Microglia-Mediated Neuroprotection, TREM2, and Alzheimer's Disease: Evidence From Optical Imaging. Biol Psychiatry 83, 377-387 (2018).
31. E. C. Damisah, A. Rai, J. Grutzendler, TREM2: Modulator of Lipid Metabolism in Microglia. Neuron 105, 759-761 (2020).
32. A. Griciuc et al., TREM2 Acts Downstream of CD33 in Modulating Microglial Pathology in Alzheimer's Disease. Neuron 103, 820-835 e827 (2019).
33. E. R. Roy et al., Type I interferon response drives neuroinflammation and synapse loss in Alzheimer disease. J Clin Invest 130, 1912-1930 (2020).
34. A. Benmamar-Badel, T. Owens, A. Wlodarczyk, Protective Microglial Subset in Development, Aging, and Disease: Lessons From Transcriptomic Studies. Front Immunol 11, 430 (2020).
35. S. Krasemann et al., The TREM2-APOE Pathway Drives the Transcriptional Phenotype of Dysfunctional Microglia in Neurodegenerative Diseases. Immunity 47, 566-581 e569 (2017).
36. D. M. Underhill, H. S. Goodridge, The many faces of ITAMs. Trends Immunol 28, 66-73 (2007).
37. C. L. Abram, C. A. Lowell, The expanding role for ITAM-based signaling pathways in immune cells. Sci STKE 2007, re2 (2007).
38. X. Zhong, B. Chen, L. Yang, Z. Yang, Molecular and physiological roles of the adaptor protein CARD9 in immunity. Cell Death Dis 9, 52 (2018).
39. Y. Zhang et al., An RNA-sequencing transcriptome and splicing database of glia, neurons, and vascular cells of the cerebral cortex. J Neurosci 34, 11929-11947 (2014).
40. S. Forner et al., Systematic phenotyping and characterization of the 5×FAD mouse model of Alzheimer's disease. Sci Data 8, 270 (2021).
41. B. C. Richard et al., Gene Dosage Dependent Aggravation of the Neurological Phenotype in the 5×FAD Mouse Model of Alzheimer's Disease. J Alzheimers Dis 45, 1223-1236 (2015).
42. S. T. Ferreira, M. V. Lourenco, M. M. Oliveira, F. G. De Felice, Soluble amyloid-beta oligomers as synaptotoxins leading to cognitive impairment in Alzheimer's disease. Front Cell Neurosci 9, 191 (2015).
43. C. Yu, E. Nwabuisi-Heath, K. Laxton, M. J. Ladu, Endocytic pathways mediating oligomeric Abeta42 neurotoxicity. Mol Neurodegener 5, 19 (2010).
44. M. E. Larson, S. E. Lesne, Soluble Abeta oligomer production and toxicity. J Neurochem 120 Suppl 1, 125-139 (2012).
45. B. R. Roberts et al., Biochemically-defined pools of amyloid-beta in sporadic Alzheimer's disease: correlation with amyloid PET. Brain 140, 1486-1498 (2017).
46. Z. Cai, M. D. Hussain, L. J. Yan, Microglia, neuroinflammation, and beta-amyloid protein in Alzheimer's disease. Int J Neurosci 124, 307-321 (2014).
47. N. J. Haughey et al., Disruption of neurogenesis by amyloid beta-peptide, and perturbed neural progenitor cell homeostasis, in models of Alzheimer's disease. J Neurochem 83, 1509-1524 (2002).
48. S. Jawhar, A. Trawicka, C. Jenneckens, T. A. Bayer, O. Wirths, Motor deficits, neuron loss, and reduced anxiety coinciding with axonal degeneration and intraneuronal Abeta aggregation in the 5×FAD mouse model of Alzheimer's disease. Neurobiol Aging 33, 196 e129-140 (2012).
49. J. H. Jhoo et al., Beta-amyloid (1-42)-induced learning and memory deficits in mice: involvement of oxidative burdens in the hippocampus and cerebral cortex. Behav Brain Res 155, 185-196 (2004).
50. H. Kadowaki et al., Amyloid beta induces neuronal cell death through ROS-mediated ASK1 activation. Cell Death Differ 12, 19-24 (2005).
51. H. Oakley et al., Intraneuronal beta-amyloid aggregates, neurodegeneration, and neuron loss in transgenic mice with five familial Alzheimer's disease mutations: potential factors in amyloid plaque formation. J Neurosci 26, 10129-10140 (2006).
52. T. Bartsch, J. Dohring, A. Rohr, O. Jansen, G. Deuschl, CA1 neurons in the human hippocampus are critical for autobiographical memory, mental time travel, and autonoetic consciousness. Proc Natl Acad Sci USA 108, 17562-17567 (2011).
53. F. Ugolini et al., Different Patterns of Neurodegeneration and Glia Activation in CA1 and CA3 Hippocampal Regions of TgCRND8 Mice. Front Aging Neurosci 10, 372 (2018).
54. J. F. Reilly et al., Amyloid deposition in the hippocampus and entorhinal cortex: quantitative analysis of a transgenic mouse model. Proc Natl Acad Sci USA 100, 4837-4842 (2003).
55. D. R. Thal et al., Sequence of Abeta-protein deposition in the human medial temporal lobe. J Neuropathol Exp Neurol 59, 733-748 (2000).
56. W. A. Eimer. R. Vassar, Neuron loss in the 5×FAD mouse model of Alzheimer's disease correlates with intraneuronal Abeta42 accumulation and Caspase-3 activation. Mol Neurodegener 8, 2 (2013).
57. C. V. Vorhees, M. T. Williams, Morris water maze: procedures for assessing spatial and related forms of learning and memory. Nat Protoc 1, 848-858 (2006).
58. J. Rogers, L. Churilov, A. J. Hannan, T. Renoir, Search strategy selection in the Morris water maze indicates allocentric map formation during learning that underpins spatial memory formation. Neurobiol Learn Mem 139, 37-49 (2017).
59. P. Yuan et al., TREM2 Haplodeficiency in Mice and Humans Impairs the Microglia Barrier Function Leading to Decreased Amyloid Compaction and Severe Axonal Dystrophy. Neuron 90, 724-739 (2016).
60. M. W. Marlatt et al., Proliferation in the Alzheimer hippocampus is due to microglia, not astroglia, and occurs at sites of amyloid deposition. Neural Plast 2014, 693851 (2014).
61. C. Condello et al., Structural heterogeneity and intersubject variability of Abeta in familial and sporadic Alzheimer's disease. Proc Natl Acad Sci USA 115, E782-E791 (2018).
62 R. Parakalan et al., Transcriptome analysis of amoeboid and ramified microglia isolated from the corpus callosum of rat brain. BMC Neurosci 13, 64 (2012).
63. J. Marschallinger et al., Lipid-droplet-accumulating microglia represent a dysfunctional and proinflammatory state in the aging brain. Nat Neurosci 23, 194-208 (2020).
64. W. Y. Wang, M. S. Tan. J. T. Yu, L. Tan, Role of pro-inflammatory cytokines released from microglia in Alzheimer's disease. Ann Transl Med 3, 136 (2015).
65. E. S. N. M. Lyra et al., Pro-inflammatory interleukin-6 signaling links cognitive impairments and peripheral metabolic alterations in Alzheimer's disease. Transl Psychiatry 11, 251 (2021).
66 B. Parajuli et al., Oligomeric amyloid beta induces IL-1beta processing via production of ROS: implication in Alzheimer's disease. Cell Death Dis 4, e975 (2013).
67. J. Ojala et al., Expression of interleukin-18 is increased in the brains of Alzheimer's disease patients. Neurobiol Aging 30, 198-209 (2009).
68. H. Hara et al., The adaptor protein CARD9 is essential for the activation of myeloid cells through ITAM-associated and Toll-like receptors. Nat Immunol 8, 619-629 (2007).
69 H. Keren-Shaul et al., A Unique Microglia Type Associated with Restricting Development of Alzheimer's Disease. Cell 169, 1276-1290 e1217 (2017).
70. A. Deczkowska et al., Disease-Associated Microglia: A Universal Immune Sensor of Neurodegeneration. Cell 173, 1073-1081 (2018).
71. O. Butovsky, H. L. Weiner, Microglial signatures and their role in health and disease. Nat Rev Neurosci 19, 622-635 (2018).
72. L. Li, X. Zi, D. Hou, Q. Tu, Kruppel-like factor 4 regulates amyloid-beta (Abeta)-induced neuroinflammation in Alzheimer's disease. Neurosci Lett 643, 131-137 (2017).
73. Z. Cheng et al., The Role of KLF4 in Alzheimer's Disease. Front Cell Neurosci 12, 325 (2018).
74. B. Kong et al., microRNA-7 Protects Against 1-Methyl-4-Phenylpyridinium Iodide-Induced Cell Apoptosis in SH-SY5Y Cells by Directly Targeting Krupple-Like Factor 4. DNA Cell Biol 35, 217-225 (2016).
75. J. Chen et al., Induction of KLF4 contributes to the neurotoxicity of MPP+in M17 cells: a new implication in Parkinson's disease. J Mol Neurosci 51, 109-117 (2013).
76. J. Kwiatek-Majkusiak et al., Relationships between typical histopathological hallmarks and the ferritin in the hippocampus from patients with Alzheimer's disease. Acta Neurobiol Exp (Wars) 75, 391-398 (2015).
77. A. M. Koorts, M. Viljoen, Ferritin and ferritin isoforms I: Structure-function relationships, synthesis, degradation and secretion. Arch Physiol Biochem 113, 30-54 (2007).
78. S. Kiani Shabestari et al., Absence of microglia promotes diverse pathologies and early lethality in Alzheimer's disease mice. Cell Rep 39, 110961 (2022).
79. A. L. Hemonnot, J. Hua, L. Ulmann, H. Hirbec, Microglia in Alzheimer Disease: Well-Known Targets and New Opportunities. Front Aging Neurosci 11, 233 (2019).
80. P. d′Errico et al., Microglia contribute to the propagation of Abeta into unaffected brain tissue. Nat Neurosci 25, 20-25 (2022).
81. H. S. Goodridge et al., Activation of the innate immune receptor Dectin-1 upon formation of a ‘phagocytic synapse’. Nature 472, 471-475 (2011).
82. H. S. Goodridge et al., Differential use of CARD9 by dectin-1 in macrophages and dendritic cells. J Immunol 182, 1146-1154 (2009).
83. R. A. Drummond, S. Saijo, Y. Iwakura, G. D. Brown, The role of Syk/CARD9 coupled C-type lectins in antifungal immunity. Eur J Immunol 41, 276-281 (2011).
84. C. Cruchaga et al., GWAS of cerebrospinal fluid tau levels identifies risk variants for Alzheimer's disease. Neuron 78, 256-268 (2013).
85. A. Inc., A Phase 2 Study to Evaluate Efficacy and Safety of AL002 in Participants With Early Alzheimer's Disease (INVOKE-2). (2020).
86. A. Inc., First in Human Study for Safety and Tolerability of AL003. (2020).
87. U. o. Oxford, Microglial Colony Stimulating Factor-1 Receptor (CSFIR) in Alzheimer's Disease (MICAD). (2019).
88. B. Kenkhuis et al., Iron loading is a prominent feature of activated microglia in Alzheimer's disease patients. Acta Neuropathol Conunun 9, 27 (2021).
89. A. McIntosh et al., Iron accumulation in microglia triggers a cascade of events that leads to altered metabolism and compromised function in APP/PSI mice. Brain Pathol 29, 606-621 (2019).
90. M. Colonna, All roads lead to CARD9. Nat Immunol 8, 554-555 (2007).
91. A. Malik et al., SYK-CARD9 Signaling Axis Promotes Gut Fungi-Mediated Inflammasome Activation to Restrict Colitis and Colon Cancer. Immunity 49, 515-530 e515 (2018).
92. R. B. Rock et al., Role of microglia in central nervous system infections. Clin Microbiol Rev 17, 942-964, table of contents (2004).
93. C. F. Hatton, C. J. A. Duncan, Microglia Are Essential to Protective Antiviral Immunity: Lessons From Mouse Models of Viral Encephalitis. Front Immunol 10, 2656 (2019).
94. R. A. Drummond et al., CARD9 (+) microglia promote antifungal immunity via IL-1beta-and CXCLI-mediated neutrophil recruitment. Nat Immunol 20, 559-570 (2019).
95. U. Puntener, S. G. Booth, V. H. Perry, J. L. Tecling, Long-term impact of systemic bacterial infection on the cerebral vasculature and microglia. J Neuroinflammation 9, 146 (2012).
96. D. Pisa, R. Alonso, A. Rabano, I. Rodal, L. Carrasco, Different Brain Regions are Infected with Fungi in Alzheimer's Disease. Sci Rep 5, 15015 (2015).
97. R. Alonso, D. Pisa, A. M. Fernandez-Fernandez, L. Carrasco, Infection of Fungi and Bacteria in Brain Tissue From Elderly Persons and Patients With Alzheimer's Disease. Front Aging Neurosci 10, 159 (2018).
98. B. Parady, Innate Immune and Fungal Model of Alzheimer's Disease. J Alzheimers Dis Rep 2, 139-152 (2018).
99. Y. Wu et al., Microglia and amyloid precursor protein coordinate control of transient Candida cerebritis with memory deficits. Nat Commun 10, 58 (2019).
100. P. W. de Groot, O. Bader, A. D. de Boer, M. Weig, N. Chauhan, Adhesins in human fungal pathogens: glue with plenty of stick. Eukaryot Cell 12, 470-481 (2013).
101. N. Shanmugam et al., Microbial functional amyloids serve diverse purposes for structure, adhesion and defence. Biophys Rev 11, 287-302 (2019).
102. R. Guerreiro, J. Bras, The age factor in Alzheimer's disease. Genome Med 7, 106 (2015).
103. D. P. Wightman et al., A genome-wide association study with 1,126,563 individuals identifies new risk loci for Alzheimer's disease. Nat Genet 53, 1276-1282 (2021).
104. M. Chin-Chan, J. Navarro-Yepes, B. Quintanilla-Vega, Environmental pollutants as risk factors for neurodegenerative disorders: Alzheimer and Parkinson diseases. Front Cell Neurosci 9, 124 (2015).
105. H. E. Ennerfelt, J. R. Lukens, The role of innate immunity in Alzheimer's disease. Immunol Rev 297, 225-246 (2020).
106. J. E. Rexach et al., Tau Pathology Drives Dementia Risk-Associated Gene Networks toward Chronic Inflammatory States and Immunosuppression. Cell Rep 33, 108398 (2020).
107. S. Da Mesquita et al., Functional aspects of meningeal lymphatics in ageing and Alzheimer's disease. Nature 560, 185-191 (2018).
108. W. B. Stine, L. Jungbauer, C. Yu, M. J. LaDu, Preparing synthetic Abeta in different aggregation states. Methods Mol Biol 670, 13-32 (2011).
109. G. T. Norris et al., Neuronal integrity and complement control synaptic material clearance by microglia after CNS injury. J Exp Med 215, 1789-1801 (2018).
110. Ennerfelt H, Frost EL, Shapiro DA, Holliday C, Zengeler KE, Voithofer G, Bolte AC, Lammert CR. Kulas JA, Ulland TK, Lukens JR. SYK coordinates neuroprotective microglial responses in neurodegenerative disease. Cell 185, 4135-4152 (2022).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

What is claimed is:

1. A method to enhance or activate protective microglial activities (such as phagocytosis of neurotoxic material) or reduce Aβ coverage and plaque volume compared to an untreated control comprising administering to a subject in need thereof an activator of CARD9 or genetically increasing expression of CARD9 in said subject.

2. The method of claim 1, wherein the CARD9 activator is a ligand for CLEC7A or CARD9 expression is increased by administering an expression vector comprising a nucleic acid sequence coding for CARD9.

3. The method of claim 1, wherein the CARD9 activator comprises a β-1,3-linked or a β-1,6-linked glucan.

4. The method of claim 1, wherein the CARD9 activator is pustulan.

5. The method of claim 1, wherein the subject has a demyelinating neuroinflammatory disease.

6. The method of claim 1, wherein the subject has is Alzheimer's Disease (AD) or Multiple Sclerosis (MS).

7. A method to preserve neuronal health or prevent neuronal loss comprising administering to a subject in need thereof an activator of CARD9 or genetically increasing expression of CARD9 in said subject.

8. The method of claim 7, wherein the CARD9 activator is a ligand for CLEC7A or CARD9 expression is increased by administering an expression vector comprising a nucleic acid sequence coding for CARD9.

9. The method of claim 7, wherein the CARD9 activator comprises a β-1,3-linked or a β-1,6-linked glucan.

10. The method of claim 7, wherein the CARD9 activator is pustulan.

11. The method of claim 7, wherein the subject has a demyelinating neuroinflammatory disease.

12. The method of claim 7, wherein the subject has is Alzheimer's Disease (AD) or Multiple Sclerosis (MS).

13. A method to prevent a decrease or generate an increase in spatial learning and/or memory comprising administering to a subject in need thereof an activator of CARD9 or genetically increasing expression of CARD9 in said subject.

14. The method of claim 13, wherein the CARD9 activator is a ligand for CLEC7A or CARD9 expression is increased by administering an expression vector comprising a nucleic acid sequence coding for CARD9.

15. The method of claim 13, wherein the CARD9 activator comprises a β-1,3-linked or a β-1,6-linked glucan.

16. The method of claim 13, wherein the CARD9 activator is pustulan.

17. The method of claim 13, wherein the subject has a demyelinating neuroinflammatory disease.

18. The method of claim 13, wherein the subject has is Alzheimer's Disease (AD) or Multiple Sclerosis (MS).


SEQUENCE LISTING (2 sequences)

SEQ ID NO: 1 - protein, 492 aa, Homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGCLAGGGSPKQPFAALHQEQVLRNPHDAGPAGLPGIGAVC

SEQ ID NO: 2 - DNA, 1802 bp, Homo sapiens
  source 1..1802: mol_type = genomic DNA
aagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtgtgctcctggacatcctgcagcggaccggccacaagggctacgtggccttcctcgagagcctggagctctactacccgcagctgtacaagaaggtcacaggcaaggagccggcccgcgtcttctccatgatcatcgacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccggtgcatggaggagaaggagatgttcgagctgcagtgcctggcactacgtaaggactccaagatgtacaaggaccgcatcgaggccatcctgctgcagatggaggaggtcgccattgagcgggaccaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctgagctccgacctggaagatggctcacccaggaggtcccaggagctctcactcccccaggacctggaggacacccagctctcagacaaaggctgccttgccggcggggggagcccgaaacagccctttgcagctctgcaccaggagcaggttttgcggaacccccatgacgcaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgca